(19) United States
(12) Patent Application Publication
(10) Pub. No.: US 2004/0137467 A1
(43) Pub. Date: Jul. 15, 2004

(54) NPC1L1 (NPC3) and methods of use thereof

(75) Inventors: Scott W. Altmann, Fanwood, NJ (US); Nicholas J. Murgolo, Millington, NJ (US); Luquan Wang, East Brunswick, NJ (US); Michael P. Graziano, Scotch Plains, NJ (US)

Correspondence Address: SCHERING-PLOUGH CORPORATION, PATENT DEPARTMENT (K-6-1, 1990), 2000 GALLOPING HILL ROAD, KENILWORTH, NJ, 07033-0530, US

(73) Assignee: Schering Corporation

(21) Appl. No.: 10/646,301

(22) Filed: Aug. 22, 2003

Related U.S. Application Data
(63) Continuation-in-part of application No. 10/621,758, filed on Jul. 17, 2003.
(60) Provisional application No. 60/397,442, filed on Jul. 19, 2002.

(51) Int. Cl. C12Q 1/68; C07H 21/04; C07K 14/705; C07K 16/18

(52) U.S. Cl. 435/6; 435/69.1; 435/320.1; 435/325; 530/350; 530/388.1; 536/23.5

(57) ABSTRACT

The present invention provides rat and mouse NPC1L1 polypeptides and polynucleotides encoding the polypeptides. Also provided are methods for detecting agonists and antagonists of NPC1L1. Inhibitors of NPC1L1 can be used for inhibiting intestinal cholesterol absorption in a subject.

This application is a continuation-in-part of U.S. patent application Ser. No. 10/621,758, filed Jul. 17, 2003 which claims the benefit of U.S. Provisional Patent Application No. 60/397,442; filed Jul. 19, 2002 each of which is herein incorporated by reference in its entirety.

### FIELD OF THE INVENTION

The present invention includes NPC1L1 polypeptides and polynucleotides which encode the polypeptides along with methods of use thereof.

### BACKGROUND OF THE INVENTION

A factor leading to development of vascular disease, a leading cause of death in industrialized nations, is elevated serum cholesterol. It is estimated that 19% of Americans between the ages of 20 and 74 years of age have high serum cholesterol. The most prevalent form of vascular disease is arteriosclerosis, a condition associated with the thickening and hardening of the arterial wall. Arteriosclerosis of the large vessels is referred to as atherosclerosis. Atherosclerosis is the predominant underlying factor in vascular disorders such as coronary artery disease, aortic aneurysm, arterial disease of the lower extremities and cerebrovascular disease.

Cholesteryl esters are a major component of atherosclerotic lesions and the major storage form of cholesterol in arterial wall cells. Formation of cholesteryl esters is also a step in the intestinal absorption of dietary cholesterol. Thus, inhibition of cholesteryl ester formation and reduction of serum cholesterol can inhibit the progression of atherosclerotic lesion formation, decrease the accumulation of cholesteryl esters in the arterial wall, and block the intestinal absorption of dietary cholesterol.

The regulation of whole-body cholesterol homeostasis in mammals and animals involves the regulation of intestinal cholesterol absorption, cellular cholesterol trafficking, dietary cholesterol and modulation of cholesterol biosynthesis, bile acid biosynthesis, steroid biosynthesis and the catabolism of the cholesterol-containing plasma lipoproteins. Regulation of intestinal cholesterol absorption has proven to be an effective means by which to regulate serum cholesterol levels. For example, a cholesterol absorption inhibitor, ezetimibe (

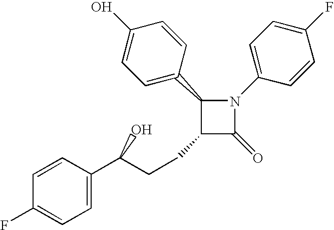

), has been shown to be effective in this regard. A pharmaceutical composition containing ezetimibe is commercially available from Merck/Schering-Plough Pharmaceuticals, Inc. under the tradename Zeita®. Identification of a gene target through which ezetimibe acts is important to understanding the process of cholesterol absorption and to the development of other, novel absorption inhibitors. The present invention addresses this need by providing a rat and a mouse homologue of human NPC1L1 (also known as NPC3; Genbank Accession No. AF192522; Davies, et al., (2000) Genomics 65(2):137-45 and Ioannou, (2000) Mol. Genet. Metab.71(1-2):175-81), an ezetimibe target.

NPC1L1 is an N-glycosylated protein comprising a YQRL (SEQ ID NO: 38) motif (i.e., a trans-golgi network to plasma membrane transport signal; see Bos, et al., (1993) EMBO J. 12:2219-2228; Humphrey, et al., (1993) J. Cell. Biol. 120:1123-1135; Ponnambalam, et al., (1994) J. Cell. Biol. 125:253-268 and Rothman, et al., (1996) Science 272:227-234) which exhibits limited tissue distribution and gastrointestinal abundance. Also, the human NPC1L1 promoter includes a Sterol Regulated Element Binding Protein 1(SREBP1) binding consensus sequence (Athanikar, et al., (1998) Proc. Natl. Acad. Sci. USA 95:4935-4940; Ericsson, et al., (1996) Proc. Natl. Acad. Sci. USA 93:945-950; Metherall, et al., (1989) J. Biol. Chem. 264:15634-15641; Smith, et al., (1990) J. Biol. Chem. 265:2306-2310; Bennett, et al., (1999) J. Biol. Chem. 274:13025-13032 and Brown, et al., (1997) Cell 89:331-340). NPC1L1 has 42% amino acid sequence homology to human NPC1 (Genbank Accession No. AF002020), a receptor responsible for Niemann-Pick C1 disease (Carstea, et al., (1997) Science 277:228-231). Niemann-Pick C1 disease is a rare genetic disorder in humans which results in accumulation of low density lipoprotein (LDL)-derived unesterified cholesterol in lysosomes (Pentchev, et al., (1994) Biochim. Biophys. Acta. 1225: 235-243 and Vanier, et al., (1991) Biochim. Biophys. Acta. 1096:328-337). In addition, cholesterol accumulates in the trans-golgi network of npc1⁻ cells, and relocation of cholesterol, to and from the plasma membrane, is delayed. NPC1 and NPC1L1 each possess 13 transmembrane spanning segments as well as a sterol-sensing domain (SSD). Several other proteins, including HMG-CoA Reductase (HMG-R), Patched (PTC) and Sterol Regulatory Element Binding Protein Cleavage-Activation Protein (SCAP), include an SSD which is involved in sensing cholesterol levels possibly by a mechanism which involves direct cholesterol binding (Gil, et al., (1985) Cell 41:249-258; Kumagai, et al., (1995) J. Biol. Chem. 270:19107-19113 and Hua, et al., (1996) Cell 87:415-426).

### SUMMARY OF THE INVENTION

The present invention includes an isolated polypeptide comprising 42 or more contiguous amino acids from an amino acid sequence selected from SEQ ID NOs: 2 and 12, preferably comprising the amino acid sequence selected from SEQ ID NOs: 2 and 12. The invention also includes an isolated polynucleotide encoding a polypeptide of SEQ ID NO: 2 or 12, preferably comprising a nucleotide sequence selected from SEQ ID NOs: 1, 5-10, 11 and 13. A recombinant vector comprising a polynucleotide of the invention is also provided along with a host cell comprising the vector.

The present invention also provides an antibody which specifically binds to NPC1L1 (e.g., mouse NPC1L1 or human NPC1L1) or any antigenic fragment thereof, preferably rat NPC1L1, more preferably a polypeptide comprising an amino acid sequence selected from SEQ ID NO: 39-42. Preferably, the antibody is a polyclonal or monoclonal antibody. Preferably, the antibody is obtained from a rabbit.

The present invention also includes a method for making an NPC1L1 polypeptide of the invention comprising culturing a host cell of the invention under conditions in which the nucleic acid in the cell which encodes the NPC1L1 polypeptide is expressed. Preferably, the method includes the step of isolating the polypeptide from the culture.

The present invention includes methods for identifying an agonist or antagonist of NPC1L1 comprising (a) contacting a host cell (e.g., chinese hamster ovary (CHO) cell, a J774 cell, a macrophage cell or a Caco2 cell) expressing a polypeptide comprising the amino acid sequence of SEQ ID NO: 2 or SEQ ID NO: 4 or SEQ ID NO: 12 or a functional fragment thereof on a cell surface, in the presence of a known amount of a detectably labeled (e.g., with ³H or ¹²⁵I) 2-azetidinone (e.g., ezetimibe), with a sample to be tested for the presence of an NPC1L1 agonist or antagonist; and (b) measuring the amount of detectably labeled 2-azetidinone (e.g., ezetimibe) specifically bound to the polypeptide; wherein an NPC1L1 agonist or antagonist in the sample is identified by measuring substantially reduced binding of the detectably labeled 2-azetidinone (e.g., ezetimibe) to the polypeptide, compared to what would be measured in the absence of such an agonist or antagonist.

Another method for identifying an agonist or antagonist of NPC1L1 is also provided. The method comprises (a) placing, in an aqueous suspension, a plurality of support particles, impregnated with a fluorescer (e.g., yttrium silicate, yttrium oxide, diphenyloxazole and polyvinyltoluene), to which a host cell (e.g., chinese hamster ovary (CHO) cell, a J774 cell, a macrophage cell or a Caco2 cell) expressing a polypeptide comprising the amino acid sequence of SEQ ID NO: 2 or SEQ ID NO: 4 or SEQ ID NO: 12 or a functional fragment thereof on a cell surface are attached; (b) adding, to the suspension, a radiolabeled (e.g., with ³H or ¹²⁵I) 2-azetidinone (e.g., ezetimibe) and a sample to be tested for the presence of an antagonist or agonist, wherein the radiolabel emits radiation energy capable of activating the fluorescer upon the binding of the 2-azetidinone (e.g., ezetimibe) to the polypeptide to produce light energy, whereas radiolabeled 2-azetidinone (e.g., ezetimibe) that does not bind to the polypeptide is, generally, too far removed from the support particles to enable the radioactive energy to activate the fluorescer; and (c) measuring the light energy emitted by the fluorescer in the suspension; wherein an NPC1L1 agonist or antagonist in the sample is identified by measuring substantially reduced light energy emission, compared to what would be measured in the absence of such an agonist or antagonist.

Also provided is a method for identifying an agonist or antagonist of NPC1L1 comprising (a) contacting a host cell (e.g., chinese hamster ovary (CHO) cell, a J774 cell, a macrophage cell or a Caco2 cell) expressing an polypeptide comprising an amino acid sequence of SEQ ID NO: 2 or SEQ ID NO: 4 or SEQ ID NO: 12 or a functional fragment thereof on a cell surface with detectably labeled (e.g., with ³H and ¹²⁵I) sterol (e.g., cholesterol) or 5α-stanol and with a sample to be tested for the presence of an antagonist or agonist; and (b) measuring the amount of detectably labeled sterol (e.g., cholesterol) or 5α-stanol in the cell; wherein an NPC1L1 antagonist in the sample is identified by measuring substantially reduced detectably labeled sterol (e.g., cholesterol) or 5α-stanol within the host cell, compared to what would be measured in the absence of such an antagonist and wherein an NPC1L1 agonist in the sample is identified by measuring substantially increased detectably labeled sterol (e.g., cholesterol) or 5α-stanol within the host cell, compared to what would be measured in the absence of such an agonist.

The present invention includes methods for inhibiting NPC1L1-mediated intestinal sterol (e.g., cholesterol) or 5α-stanol uptake, in a subject, by administering a substance identified by the screening methods described herein to the subject. Such substances include compounds such as small molecule antagonists of NPC1L1 other than ezetimibe. Also contemplated are methods for antagonizing NPC1L1-mediated sterol (e.g., cholesterol) or 5α-stanol absorption by administering anti-NPC1L1 antibodies. NPC1L1-mediated absorption of sterol (e.g., cholesterol) or 5α-stanol can also be antagonized by any method which reduces expression of NPC1L1 in an organism. For example, NPC1L1 expression can be reduced by introduction of anti-sense NPC1L1 mRNA into a cell of an organism or by genetic mutation of the NPC1L1 gene in an organism (e.g., by complete knockout, disruption, truncation or by introduction of one or more point mutations).

Also included in the present invention is a mutant mouse comprising a homozygous or heterozygous mutation (e.g., disruption, truncation, one or more point mutations, knock out) of endogenous, chromosomal NPC1L1 wherein, preferably, the mouse does not produce any functional NPC1L1 protein. Preferably, the mutant mouse, lacking functional NPC1L1, exhibits reduced intestinal sterol (e.g., cholesterol) or 5α-stanol absorption and/or reduced serum sterol (e.g., cholesterol) or 5α-stanol. Preferably, in the mutant mouse chromosome, the region of NPC1L1 (SEQ ID NO: 45) deleted is from nucleotide 790 to nucleotide 998. Any offspring or progeny of a parent NPC1L1 mutant mouse (i.e., npc1l1) of the invention which has inherited an npc1l1 mutant allele is also part of the present invention.

The scope of the present invention also includes a method for screening a sample for an intestinal sterol (e.g., cholesterol) or 5α-stanol absorption antagonist comprising (a) feeding a sterol (e.g., cholesterol) or 5α-stanol-containing substance (eg., comprising radiolabeled cholesterol, such as ¹⁴C-cholesterol or ³H-cholesterol) to a first and second mouse comprising a functional NPC1L1 gene and to a third, mutant mouse lacking a functional NPC1L1; (b) administering the sample to the first mouse comprising a functional NPC1L1 but not to the second mouse; (c) measuring the amount of sterol (e.g., cholesterol) or 5α-stanol absorption in the intestine of said first, second and third mouse (e.g., by measuring serum cholesterol); and (d) comparing the levels of intestinal sterol (e.g., cholesterol) or 5α-stanol absorption in each mouse; wherein the sample is determined to contain the intestinal sterol (e.g., cholesterol) or 5α-stanol absorption antagonist when the level of intestinal sterol (e.g., cholesterol) or 5α-stanol absorption in the first mouse is less than the amount of intestinal sterol (e.g., cholesterol) or 5α-stanol absorption in the second mouse.

The present invention also encompasses a kit comprising (a) a 2-azetidinone (e.g., ezetimibe) in a pharmaceutical dosage form (e.g., a pill or tablet comprising 10 mg 2-azetidinone (e.g., ezetimibe)); and (b) information, for example in the form of an insert, indicating that NPC1L1 is a target of ezetimibe. The kit may also include simvastatin in a pharmaceutical dosage form (e.g., a pill or tablet comprising 5 mg, 10 mg, 20 mg, 40 mg or 80 mg simvastatin). The simvastatin in pharmaceutical dosage form and the ezetimibe in pharmaceutical dosage form can be associated in a single pill or tablet or in separate pills or tablets.

### DETAILED DESCRIPTION OF THE INVENTION

The present invention includes an NPC1L1 polypeptide from rat and from mouse along with polynucleotides encoding the respective polypeptides. Preferably, the rat NPC1L1 polypeptide comprises the amino acid sequence set forth in SEQ ID NO: 2 and the mouse NPC1L1 polypeptide comprises the amino acid sequence set forth in SEQ ID NO.12. The rat NPC1L1 polynucleotide of SEQ ID NO:1 or 10encodes the rat NPC1L1 polypeptide. The mouse NPC1L1 polynucleotide of SEQ ID NO:11 or 13 encodes the mouse NPC1L1 polypeptide.

The present invention includes any polynucleotide or polypeptide comprising a nucleotide or amino acid sequence referred to, below, in Table 1.

TABLE 1
Polynucleotides and Polypeptides of the Invention.

| Polynucleotide or Polypeptide | Sequence Identifier |
|---|---|
| Rat NPC1L1 polynucleotide | SEQ ID NO: 1 |
| Rat NPC1L1 polypeptide | SEQ ID NO: 2 |
| Human NPC1L1 polynucleotide | SEQ ID NO: 3 |
| Human NPC1L1 polypeptide | SEQ ID NO: 4 |
| Rat NPC1L1 expressed sequence tag | SEQ ID NO: 5 |
| 603662080F1 (partial sequence) | |
| Rat NPC1L1 expressed sequence tag | SEQ ID NO: 6 |
| 603665037F1 (partial sequence) | |
| Rat NPC1L1 expressed sequence tag | SEQ ID NO: 7 |
| 604034587F1 (partial sequence) | |
| EST 603662080F1 with downstream | SEQ ID NO: 8 |
| sequences added | |
| EST 603662080F1 with upstream and | SEQ ID NO: 9 |
| downstream sequences added | |
| Back-translated polynucleotide sequence of | SEQ ID NO: 10 |
| rat NPC1L1 | |
| Mouse NPC1L1 polynucleotide | SEQ ID NO: 11 |
| Mouse NPC1L1 polypeptide | SEQ ID NO: 12 |
| Back-translated polynucleotide sequence of | SEQ ID NO: 13 |
| mouse NPC1L1 | |

A human NPC1L1 is also disclosed under Genbank Accession Number AF192522. As discussed below, the nucleotide sequence of the rat NPC1L1 set forth in SEQ ID NO: 1 was obtained from an expressed sequence tag (EST) from a rat jejunum enterocyte cDNA library. SEQ ID NOs: 5-7 include partial nucleotide sequences of three independent cDNA clones. The downstream sequence of the SEQ ID NO: 5 EST (603662080F1) were determined; the sequencing data from these experiments are set forth in SEQ ID NO: 8. The upstream sequences were also determined; these data are set forth in SEQ ID NO: 9.

SEQ ID NOs: 43 and 44 are the nucleotide and amino acid sequence, respectively, of human NPC1L1 which is disclosed under Genbank Accession No.: AF192522 (see Davies, et al., (2000) Genomics 65(2):137-45).

SEQ ID NO: 45 is the nucleotide sequence of a mouse NPC1L1 which is disclosed under Genbank Accession No. AK078947.

NPC1L1 mediates intestinal sterol (e.g., cholesterol) or 5α-stanol absorption. Inhibition of NPC1L1 in a patient is a useful method for reducing intestinal sterol (e.g., cholesterol) or 5α-stanol absorption and serum sterol (e.g., cholesterol) or 5α-stanol in the patient. Reducing the level of intestinal sterol (e.g., cholesterol) or 5α-stanol absorption and serum sterol (e.g., cholesterol) or 5α-stanol in a patient is a useful way in which to treat or prevent the occurrence of atherosclerosis, particularly diet-induced atherosclerosis.

### Molecular Biology

In accordance with the present invention there may be employed conventional molecular biology, microbiology, and recombinant DNA techniques within the skill of the art. Such techniques are explained fully in the literature. See, e.g., Sambrook, Fritsch & Maniatis, Molecular Cloning: A Laboratory Manual, Second Edition (1989) Cold Spring Harbor Laboratory Press, Cold Spring Harbor, N.Y. (herein “Sambrook, et al., 1989”); DNA Cloning: A Practical Approach, Volumes I and II (D. N. Glover ed. 1985); Oligonucleotide Synthesis (M. J. Gait ed. 1984); Nucleic Acid Hybridization (B. D. Hames & S. J. Higgins eds. (1985)); Transcription And Translation (B. D. Hames & S. J. Higgins, eds. (1984)); Animal Cell Culture (R. I. Freshney, ed. (1986)); Immobilized Cells And Enzymes (IRL Press, (1986)); B. Perbal, A Practical Guide To Molecular Cloning (1984); F. M. Ausubel, et al. (eds.), Current Protocols in Molecular Biology, John Wiley & Sons, Inc. (1994).

The back-translated sequences of SEQ ID NO: 10 and of SEQ ID NO: 13 uses the single-letter code shown in Table 1 of Annex C, Appendix 2 of the PCT Administrative Instruction in the Manual of Patent Examination Procedure.

A “polynucleotide”, “nucleic acid” or “nucleic acid molecule” may refer to the phosphate ester polymeric form of ribonucleosides (adenosine, guanosine, uridine or cytidine; “RNA molecules”) or deoxyribonucleosides (deoxyadenosine, deoxyguanosine, deoxythymidine, or deoxycytidine; “DNA molecules”), or any phosphoester analogs thereof, such as phosphorothioates and thioesters, in single stranded form, double-stranded form or otherwise.

A “polynucleotide sequence”, “nucleic acid sequence” or “nucleotide sequence” is a series of nucleotide bases (also called “nucleotides”) in a nucleic acid, such as DNA or RNA, and means any chain of two or more nucleotides.

A “coding sequence” or a sequence “encoding” an expression product, such as a RNA, polypeptide, protein, or enzyme, is a nucleotide sequence that, when expressed, results in production of the product.

The term “gene” means a DNA sequence that codes for or corresponds to a particular sequence of ribonucleotides or amino acids which comprise all or part of one or more RNA molecules, proteins or enzymes, and may or may not include regulatory DNA sequences, such as promoter sequences, which determine, for example, the conditions under which the gene is expressed. Genes may be transcribed from DNA to RNA which may or may not be translated into an amino acid sequence.

The present invention includes nucleic acid fragments of any of SEQ ID NOs: 1, 5-11 or 13. A nucleic acid “fragment” includes at least about 30 (e.g., 31, 32, 33, 34), preferably at least about 35 (e.g, 25, 26, 27, 28, 29, 30, 31, 32, 33 or 34), more preferably at least about 45 (e.g., 35, 36, 37, 38, 39, 40, 41, 42, 43 or 44), and most preferably at least about 126 or more contiguous nucleotides (e.g., 130, 131, 132, 133, 134, 135, 136, 137, 138, 139, 140, 150, 160, 170, 180, 190, 200, 300, 400, 500, 1000 or 1200) from any of SEQ ID NOs: 1, 5-11 or 13.

The present invention also includes nucleic acid fragments consisting of at least about 7 (e.g., 9, 12, 17, 19), preferably at least about 20 (e.g., 30, 40, 50, 60), more preferably about 70 (e.g., 80, 90, 95), yet more preferably at least about 100 (e.g., 105, 110, 114) and even more preferably at least about 115 (e.g., 117, 119, 120, 122, 124, 125, 126) contiguous nucleotides from any of SEQ ID NOs: 1, 5-11 or 13.

As used herein, the term “oligonucleotide” refers to a nucleic acid, generally of no more than about 100 nucleotides (e.g., 30, 40, 50, 60, 70, 80, or 90), that may be hybridizable to a genomic DNA molecule, a cDNA molecule, or an mRNA molecule encoding a gene, mRNA, cDNA, or other nucleic acid of interest. Oligonucleotides can be labeled, e.g., by incorporation of ³²P-nucleotides, ³H-nucleotides, ¹⁴C-nucleotides, ³⁵S-nucleotides or nucleotides to which a label, such as biotin, has been covalently conjugated. In one embodiment, a labeled oligonucleotide can be used as a probe to detect the presence of a nucleic acid. In another embodiment, oligonucleotides (one or both of which may be labeled) can be used as PCR primers, either for cloning full length or a fragment of the gene, or to detect the presence of nucleic acids. Generally, oligonucleotides are prepared synthetically, preferably on a nucleic acid synthesizer.

A “protein sequence”, “peptide sequence” or “polypeptide sequence” or “amino acid sequence” may refer to a series of two or more amino acids in a protein, peptide or polypeptide.

“Protein”, “peptide” or “polypeptide” includes a contiguous string of two or more amino acids. Preferred peptides of the invention include those set forth in any of SEQ ID NOs: 2 or 12 as well as variants and fragments thereof. Such fragments preferably comprise at least about 10 (e.g., 11, 12, 13, 14, 15, 16, 17, 18 or 19), more preferably at least about 20 (e.g., 21, 22, 23, 24, 25, 26, 27, 28, 29, 30, 35, 40), and yet more preferably at least about 42 (e.g., 43, 44, 45, 46, 47, 48, 49, 50, 60, 70, 80, 90, 100, 110, 120 or 130) or more contiguous amino acid residues from any of SEQ ID NOs: 2 or 12.

The present invention also includes polypeptides, preferably antigenic polypeptides, consisting of at least about 7 (e.g., 9, 10, 13, 15, 17, 19), preferably at least about 20 (e.g., 22, 24, 26, 28), yet more preferably at least about 30 (e.g., 32, 34, 36, 38) and even more preferably at least about 40 (e.g., 41, 42) contiguous amino acids from any of SEQ ID NOs: 2 or 12.

The polypeptides of the invention can be produced by proteolytic cleavage of an intact peptide, by chemical synthesis or by the application of recombinant DNA technology and are not limited to polypeptides delineated by proteolytic cleavage sites. The polypeptides, either alone or cross-linked or conjugated to a carrier molecule to render them more immunogenic, are useful as antigens to elicit the production of antibodies and fragments thereof. The antibodies can be used, e.g., in immunoassays for immunoaffinity purification or for inhibition of NPC1L1, etc.

The terms “isolated polynucleotide” or “isolated polypeptide” include a polynucleotide (e.g., RNA or DNA molecule, or a mixed polymer) or a polypeptide, respectively, which are partially or fully separated from other components that are normally found in cells or in recombinant DNA expression systems. These components include, but are not limited to, cell membranes, cell walls, ribosomes, polymerases, serum components and extraneous genomic sequences.

An isolated polynucleotide or polypeptide will, preferably, be an essentially homogeneous composition of molecules but may contain some heterogeneity.

“Amplification” of DNA as used herein may denote the use of polymerase chain reaction (PCR) to increase the concentration of a particular DNA sequence within a mixture of DNA sequences. For a description of PCR see Saiki, et al., Science (1988) 239:487.

The term “host cell” includes any cell of any organism that is selected, modified, transfected, transformed, grown, or used or manipulated in any way, for the production of a substance by the cell, for example the expression or replication, by the cell, of a gene, a DNA or RNA sequence or a protein. Preferred host cells include chinese hamster ovary (CHO) cells, murine macrophage J774 cells or any other macrophage cell line and human intestinal epithelial Caco2 cells.

The nucleotide sequence of a nucleic acid may be determined by any method known in the art (e.g., chemical sequencing or enzymatic sequencing). “Chemical sequencing” of DNA includes methods such as that of Maxam and Gilbert (1977) (Proc. Natl. Acad. Sci. USA 74:560), in which DNA is randomly cleaved using individual base-specific reactions. “Enzymatic sequencing” of DNA includes methods such as that of Sanger (Sanger, et al., (1977) Proc. Natl. Acad. Sci. USA 74:5463).

The nucleic acids herein may be flanked by natural regulatory (expression control) sequences, or may be associated with heterologous sequences, including promoters, internal ribosome entry sites (IRES) and other ribosome binding site sequences, enhancers, response elements, suppressors, signal sequences, polyadenylation sequences, introns, 5′- and 3′-non-coding regions, and the like.

In general, a “promoter” or “promoter sequence” is a DNA regulatory region capable of binding an RNA polymerase in a cell (e.g., directly or through other promoter-bound proteins or substances) and initiating transcription of a coding sequence. A promoter sequence is, in general, bounded at its 3′ terminus by the transcription initiation site and extends upstream (5′ direction) to include the minimum number of bases or elements necessary to initiate transcription at any level. Within the promoter sequence may be found a transcription initiation site (conveniently defined, for example, by mapping with nuclease S1), as well as protein binding domains (consensus sequences) responsible for the binding of RNA polymerase. The promoter may be operably associated with other expression control sequences, including enhancer and repressor sequences or with a nucleic acid of the invention. Promoters which may be used to control gene expression include, but are not limited to, cytomegalovirus (CMV) promoter (U.S. Pat. Nos. 5,385,839 and 5,168,062), the SV40 early promoter region (Benoist, et al., (1981) Nature 290:304-310), the promoter contained in the 3′ long terminal repeat of Rous sarcoma virus (Yamamoto, et al., (1980) Cell 22:787-797), the herpes thymidine kinase promoter (Wagner, et al., (1981) Proc. Natl. Acad. Sci. USA 78:1441-1445), the regulatory sequences of the metallothionein gene (Brinster, et al., (1982) Nature 296:39-42); prokaryotic expression vectors such as the β-lactamase promoter (Villa-Komaroff, et al., (1978) Proc. Natl. Acad. Sci. USA 75:3727-3731), or the tac promoter (DeBoer, et al., (1983) Proc. Natl. Acad. Sci. USA 80:21-25); see also “Useful proteins from recombinant bacteria” in Scientific American (1980) 242:74-94; and promoter elements from yeast or other fungi such as the Gal 4 promoter, the ADC (alcohol dehydrogenase) promoter, PGK (phosphoglycerol kinase) promoter or the alkaline phosphatase promoter.

A coding sequence is “under the control of”, “functionally associated with” or “operably associated with” transcriptional and translational control sequences in a cell when the sequences direct RNA polymerase mediated transcription of the coding sequence into RNA, preferably mRNA, which then may be RNA spliced (if it contains introns) and, optionally, translated into a protein encoded by the coding sequence.

The terms “express” and “expression” mean allowing or causing the information in a gene, RNA or DNA sequence to become manifest; for example, producing a protein by activating the cellular functions involved in transcription and translation of a corresponding gene. A DNA sequence is expressed in or by a cell to form an “expression product” such as an RNA (e.g., mRNA) or a protein. The expression product itself may also be said to be “expressed” by the cell.

The term “transformation” means the introduction of a nucleic acid into a cell. The introduced gene or sequence may be called a “clone”. A host cell that receives the introduced DNA or RNA has been “transformed” and is a “transformant” or a “clone.” The DNA or RNA introduced to a host cell can come from any source, including cells of the same genus or species as the host cell, or from cells of a different genus or species.

The term “vector” includes a vehicle (e.g., a plasmid) by which a DNA or RNA sequence can be introduced into a host cell, so as to transform the host and, optionally, promote expression and/or replication of the introduced sequence.

Vectors that can be used in this invention include plasmids, viruses, bacteriophage, integratable DNA fragments, and other vehicles that may facilitate introduction of the nucleic acids into the genome of the host. Plasmids are the most commonly used form of vector but all other forms of vectors which serve a similar function and which are, or become, known in the art are suitable for use herein. See, e.g., Pouwels, et al., Cloning Vectors: A Laboratory Manual 1985 and Supplements, Elsevier, N.Y., and Rodriguez et al. (eds.), Vectors: A Survey of Molecular Cloning Vectors and Their Uses, 1988, Buttersworth, Boston, Mass.

The term “expression system” means a host cell and compatible vector which, under suitable conditions, can express a protein or nucleic acid which is carried by the vector and introduced to the host cell. Common expression systems include E. coli host cells and plasmid vectors, insect host cells and Baculovirus vectors, and mammalian host cells and vectors.

Expression of nucleic acids encoding the NPC1L1 polypeptides of this invention can be carried out by conventional methods in either prokaryotic or eukaryotic cells. Although E. coli host cells are employed most frequently in prokaryotic systems, many other bacteria, such as various strains of Pseudomonas and Bacillus, are known in the art and can be used as well. Suitable host cells for expressing nucleic acids encoding the NPC1L1 polypeptides include prokaryotes and higher eukaryotes. Prokaryotes include both gram-negative and gram-positive organisms, e.g., E. coli and B. subtilis. Higher eukaryotes include established tissue culture cell lines from animal cells, both of non-mammalian origin, e.g., insect cells, and birds, and of mammalian origin, e.g., human, primates, and rodents.

Prokaryotic host-vector systems include a wide variety of vectors for many different species. A representative vector for amplifying DNA is pBR322 or many of its derivatives (e.g., pUC18 or 19). Vectors that can be used to express the NPC1L1 polypeptides include, but are not limited to, those containing the lac promoter (pUC-series); trp promoter (pBR322-trp); Ipp promoter (the pIN-series); lambda-pP or pR promoters (pOTS); or hybrid promoters such as ptac (pDR540). See Brosius et al., “Expression Vectors Employing Lambda-, trp-, lac-, and Ipp-derived Promoters”, in Rodriguez and Denhardt (eds.) Vectors: A Survey of Molecular Cloning Vectors and Their Uses, 1988, Buttersworth, Boston, pp. 205-236. Many polypeptides can be expressed, at high levels, in an E.coli/T7 expression system as disclosed in U.S. Pat. Nos. 4,952,496, 5,693,489 and 5,869,320 and in Davanloo, P., et al., (1984) Proc. Natl. Acad. Sci. USA 81: 2035-2039; Studier, F. W., et al., (1986) J. Mol. Biol. 189: 113-130; Rosenberg, A. H., et al., (1987) Gene 56: 125-135; and Dunn, J. J., et al., (1988) Gene 68: 259.

Higher eukaryotic tissue culture cells may also be used for the recombinant production of the NPC1L1 polypeptides of the invention. Although any higher eukaryotic tissue culture cell line might be used, including insect baculovirus expression systems, mammalian cells are preferred. Transformation or transfection and propagation of such cells have become a routine procedure. Examples of useful cell lines include HeLa cells, chinese hamster ovary (CHO) cell lines, J774 cells, Caco2 cells, baby rat kidney (BRK) cell lines, insect cell lines, bird cell lines, and monkey (COS) cell lines. Expression vectors for such cell lines usually include an origin of replication, a promoter, a translation initiation site, RNA splice sites (if genomic DNA is used), a polyadenylation site, and a transcription termination site. These vectors also, usually, contain a selection gene or amplification gene. Suitable expression vectors may be plasmids, viruses, or retroviruses carrying promoters derived, e.g., from such sources as adenovirus, SV40, parvoviruses, vaccinia virus, or cytomegalovirus. Examples of expression vectors include pCR®3.1, pCDNA1, pCD (Okayama, et al., (1985) Mol. Cell Biol. 5:1136), pMC1neo Poly-A (Thomas, et al., (1987) Cell 51:503), pREP8, pSVSPORT and derivatives thereof, and baculovirus vectors such as pAC373 or pAC610. One embodiment of the invention includes membrane bound NPC1L1. In this embodiment, NPC1L1 can be expressed in the cell membrane of a eukaryotic cell and the membrane bound protein can be isolated from the cell by conventional methods which are known in the art.

The present invention also includes fusions which include the NPC1L1 polypeptides and NPC1L1 polynucleotides of the present invention and a second polypeptide or polynucleotide moiety, which may be referred to as a “tag”. The fusions of the present invention may comprise any of the polynucleotides or polypeptides set forth in Table 1 or any subsequence or fragment thereof (discussed above). The fused polypeptides of the invention may be conveniently constructed, for example, by insertion of a polynucleotide of the invention or fragment thereof into an expression vector. The fusions of the invention may include tags which facilitate purification or detection. Such tags include glutathione-S-transferase (GST), hexahistidine (His6) tags, maltose binding protein (MBP) tags, haemagglutinin (HA) tags, cellulose binding protein (CBP) tags and myc tags. Detectable tags such as ³²p, ³⁵s, ³H, ^{99m}Tc, ¹²³I, ¹¹¹In, ⁶⁸Ga, ¹⁸F, ¹²⁵I, ¹³¹I, ^{113m}In, ⁷⁶Br, ⁶⁷Ga, ^{99m}Tc, ¹²³I, ¹¹¹In and ⁶⁸Ga may also be used to label the polypeptides and polynucleotides of the invention. Methods for constructing and using such fusions are very conventional and well known in the art.

Modifications (e.g., post-translational modifications) that occur in a polypeptide often will be a function of how it is made. For polypeptides made by expressing a cloned gene in a host, for instance, the nature and extent of the modifications, in large part, will be determined by the host cell's post-translational modification capacity and the modification signals present in the polypeptide amino acid sequence. For instance, as is well known, glycosylation often does not occur in bacterial hosts such as E. coli. Accordingly, when glycosylation is desired, a polypeptide can be expressed in a glycosylating host, generally a eukaryotic cell. Insect cells often carry out post-translational glycosylations which are similar to those of mammalian cells. For this reason, insect cell expression systems have been developed to express, efficiently, mammalian proteins having native patterns of glycosylation. An insect cell which may be used in this invention is any cell derived from an organism of the class Insecta. Preferably, the insect is Spodoptera fruigiperda (Sf9 or Sf21) or Trichoplusia ni (High 5). Examples of insect expression systems that can be used with the present invention, for example to produce NPC1L1 polypeptide, include Bac-To-Bac (Invitrogen Corporation, Carlsbad, Calif.) or Gateway (Invitrogen Corporation, Carlsbad, Calif.). If desired, deglycosylation enzymes can be used to remove carbohydrates attached during production in eukaryotic expression systems.

Other modifications may also include addition of aliphatic esters or amides to the polypeptide carboxyl terminus. The present invention also includes analogs of the NPC1L1 polypeptides which contain modifications, such as incorporation of unnatural amino acid residues, or phosphorylated amino acid residues such as phosphotyrosine, phosphoserine or phosphothreonine residues. Other potential modifications include sulfonation, biotinylation, or the addition of other moieties. For example, the NPC1L1 polypeptides of the invention may be appended with a polymer which increases the half-life of the peptide in the body of a subject. Preferred polymers include polyethylene glycol (PEG) (e.g., PEG with a molecular weight of 2 kDa, 5 kDa, 10 kDa, 12 kDa, 20 kDa, 30 kDa and 40 kDa), dextran and monomethoxypolyethylene glycol (mPEG).

The peptides of the invention may also be cyclized. Specifically, the amino- and carboxy-terminal residues of an NPC1L1 polypeptide or two internal residues of an NPC1L1 polypeptide of the invention can be fused to create a cyclized peptide. Methods for cyclizing peptides are conventional and very well known in the art; for example see Gurrath, et al., (1992) Eur. J. Biochem. 210:911-921.

The present invention contemplates any superficial or slight modification to the amino acid or nucleotide sequences which correspond to the polypeptides of the invention. In particular, the present invention contemplates sequence conservative variants of the nucleic acids which encode the polypeptides of the invention. “Sequence-conservative variants” of a polynucleotide sequence are those in which a change of one or more nucleotides in a given codon results in no alteration in the amino acid encoded at that position. Function-conservative variants of the polypeptides of the invention are also contemplated by the present invention. “Function-conservative variants” are those in which one or more amino acid residues in a protein or enzyme have been changed without altering the overall conformation and function of the polypeptide, including, but, by no means, limited to, replacement of an amino acid with one having similar properties. Amino acids with similar properties are well known in the art. For example, polar/hydrophilic amino acids which may be interchangeable include asparagine, glutamine, serine, cysteine, threonine, lysine, arginine, histidine, aspartic acid and glutamic acid; nonpolar/hydrophobic amino acids which may be interchangeable include glycine, alanine, valine, leucine, isoleucine, proline, tyrosine, phenylalanine, tryptophan and methionine; acidic amino acids which may be interchangeable include aspartic acid and glutamic acid and basic amino acids which may be interchangeable include histidine, lysine and arginine.

The present invention includes polynucleotides encoding rat or mouse NPC1L1 and fragments thereof as well as nucleic acids which hybridize to the polynucleotides. Preferably, the nucleic acids hybridize under low stringency conditions, more preferably under moderate stringency conditions and most preferably under high stringency conditions. A nucleic acid molecule is “hybridizable” to another nucleic acid molecule, such as a cDNA, genomic DNA, or RNA, when a single stranded form of the nucleic acid molecule can anneal to the other nucleic acid molecule under the appropriate conditions of temperature and solution ionic strength (see Sambrook, et al., supra). The conditions of temperature and ionic strength determine the “stringency” of the hybridization. Typical low stringency hybridization conditions are 55° C., 5×SSC, 0.1% SDS, 0.25% milk, and no formamide at 42° C.; or 30% formamide, 5×SSC, 0.5% SDS at 42° C. Typical, moderate stringency hybridization conditions are similar to the low stringency conditions except the hybridization is carried out in 40% formamide, with 5× or 6×SSC at 42° C. High stringency hybridization conditions are similar to low stringency conditions except the hybridization conditions are carried out in 50% formamide, 5× or 6×SSC and, optionally, at a higher temperature (e.g., higher than 42° C.: 57° C., 59° C., 60° C., 62° C., 63° C., 65° C. or 68 ° C.). In general, SSC is 0.15M NaCl and 0.015M Na-citrate. Hybridization requires that the two nucleic acids contain complementary sequences, although, depending on the stringency of the hybridization, mismatches between bases are possible. The appropriate stringency for hybridizing nucleic acids depends on the length of the nucleic acids and the degree of complementation, variables well known in the art. The greater the degree of similarity or homology between two nucleotide sequences, the higher the stringency under which the nucleic acids may hybridize. For hybrids of greater than 100 nucleotides in length, equations for calculating the melting temperature have been derived (see Sambrook, et al., supra, 9.50-9.51). For hybridization with shorter nucleic acids, i.e., oligonucleotides, the position of mismatches becomes more important, and the length of the oligonucleotide determines its specificity (see Sambrook, et al., supra).

Also included in the present invention are polynucleotides comprising nucleotide sequences and polypeptides comprising amino acid sequences which are at least about 70% identical, preferably at least about 80% identical, more preferably at least about 90% identical and most preferably at least about 95% identical (e.g., 95%, 96%, 97%, 98%, 99%, 100%) to the reference rat NPC1L1 nucleotide (e.g., any of SEQ ID NOs: 1 or 5-10) and amino acid sequences (e.g., SEQ ID NO: 2) or the mouse NPC1L1 nucleotide (e.g., any of SEQ ID NOs: 11 or 13) and amino acids sequences (e.g., SEQ ID NO: 12), when the comparison is performed by a BLAST algorithm wherein the parameters of the algorithm are selected to give the largest match between the respective sequences over the entire length of the respective reference sequences. Polypeptides comprising amino acid sequences which are at least about 70% similar, preferably at least about 80% similar, more preferably at least about 90% similar and most preferably at least about 95% similar (e.g., 95%, 96%, 97%, 98%, 99%, 100%) to the reference rat NPC1L1 amino acid sequence of SEQ ID NO: 2 or the mouse NPC1L1 amino acid sequence of SEQ ID NO: 12, when the comparison is performed with a BLAST algorithm wherein the parameters of the algorithm are selected to give the largest match between the respective sequences over the entire length of the respective reference sequences, are also included in the present invention.

Sequence identity refers to exact matches between the nucleotides or amino acids of two sequences which are being compared. Sequence similarity refers to both exact matches between the amino acids of two polypeptides which are being compared in addition to matches between nonidentical, biochemically related amino acids. Biochemically related amino acids which share similar properties and may be interchangeable are discussed above.

The following references regarding the BLAST algorithm are herein incorporated by reference: BLAST ALGORITHMS: Altschul, S. F., et al., (1990) J. Mol. Biol. 215:403-410; Gish, W., et al., (1993) Nature Genet. 3:266-272; Madden, T. L., et al., (1996) Meth. Enzymol. 266:131-141; Altschul, S. F., et al., (1997) Nucleic Acids Res. 25:3389-3402; Zhang, J., et al., (1997) Genome Res. 7:649-656; Wootton, J. C., et al., (1993) Comput. Chem. 17:149-163; Hancock, J. M., et al., (1994) Comput. Appl. Biosci. 10:67-70; ALIGNMENT SCORING SYSTEMS: Dayhoff, M. O., et al., “A model of evolutionary change in proteins.” in Atlas of Protein Sequence and Structure, (1978) vol. 5, suppl. 3. M. O. Dayhoff (ed.), pp. 345-352, Natl. Biomed. Res. Found., Washington, D.C.; Schwartz, R. M., et al., “Matrices for detecting distant relationships.” in Atlas of Protein Sequence and Structure, (1978) vol. 5, suppl. 3.” M. O. Dayhoff(ed.), pp.353-358, Natl. Biomed. Res. Found., Washington, D.C.; Altschul, S. F., (1991) J. Mol. Biol. 219:555-565; States, D. J., et al., (1991) Methods 3:66-70; Henikoff, S., et al., (1992) Proc. Natl. Acad. Sci. USA 89:10915-10919; Altschul, S. F., et al., (1993) J. Mol. Evol. 36:290-300; ALIGNMENT STATISTICS: Karlin, S., et al., (1990) Proc. Natl. Acad. Sci. USA 87:2264-2268; Karlin, S., et al., (1993) Proc. Natl. Acad. Sci. USA 90:5873-5877; Dembo, A., et al., (1994) Ann. Prob. 22:2022-2039; and Altschul, S. F. “Evaluating the statistical significance of multiple distinct local alignments.” in Theoretical and Computational Methods in Genome Research (S. Suhai, ed.), (1997) pp. 1-14, Plenum, New York.

### Protein Purification

The proteins, polypeptides and antigenic fragments of this invention can be purified by standard methods, including, but not limited to, salt or alcohol precipitation, affinity chromatography (e.g., used in conjunction with a purification tagged NPC1L1 polypeptide as discussed above), preparative disc-gel electrophoresis, isoelectric focusing, high pressure liquid chromatography (HPLC), reversed-phase HPLC, gel filtration, cation and anion exchange and partition chromatography, and countercurrent distribution. Such purification methods are well known in the art and are disclosed, e.g., in “Guide to Protein Purification”, Methods in Enzymology, Vol. 182, M. Deutscher, Ed., 1990, Academic Press, New York, N.Y.

Purification steps can be followed by performance of assays for receptor binding activity as described below. Particularly where an NPC1L1 polypeptide is being isolated from a cellular or tissue source, it is preferable to include one or more inhibitors of proteolytic enzymes in the assay system, such as phenylmethanesulfonyl fluoride (PMSF), Pefabloc SC, pepstatin, leupeptin, chymostatin and EDTA.

### Antibody Molecules

Antigenic (including immunogenic) fragments of the NPC1L1 polypeptides of the invention are within the scope of the present invention (e.g., 42 or more contiguous amino acids from SEQ ID NO: 2, 4 or 12). The antigenic peptides may be useful, inter alia, for preparing antibody molecules which recognize NPC1L1. Anti-NPC1L1 antibody molecules are useful NPC1L1 antagonists.

An antigen is any molecule that can bind specifically to an antibody. Some antigens cannot, by themselves, elicit antibody production. Those that can induce antibody production are immunogens.

Preferably, anti-NPC1L1 antibodies recognize an antigenic peptide comprising an amino acid sequence selected from SEQ ID NOs: 39-42 (e.g., an antigen derived from rat NPC1L1). More preferably, the antibody is A0715, A0716, A0717, A0718, A0867, A0868, A1801 or Al802.

The term “antibody molecule” includes, but is not limited to, antibodies and fragments (preferably antigen-binding fragments) thereof. The term includes monoclonal antibodies, polyclonal antibodies, bispecific antibodies, Fab antibody fragments, F(ab)₂ antibody fragments, Fv antibody fragments (e.g., V_{H} or V_{L}), single chain Fv antibody fragments and dsFv antibody fragments. Furthermore, the antibody molecules of the invention may be fully human antibodies, mouse antibodies, rat antibodies, rabbit antibodies, goat antibodies, chicken antibodies, humanized antibodies or chimeric antibodies.

Although it is not always necessary, when NPC1L1 polypeptides are used as antigens to elicit antibody production in an immunologically competent host, smaller antigenic fragments are, preferably, first rendered more immunogenic by cross-linking or concatenation, or by coupling to an immunogenic carrier molecule (i.e., a macromolecule having the property of independently eliciting an immunological response in a host animal, such as diptheria toxin or tetanus). Cross-linking or conjugation to a carrier molecule may be required because small polypeptide fragments sometimes act as haptens (molecules which are capable of specifically binding to an antibody but incapable of eliciting antibody production, i.e., they are not immunogenic). Conjugation of such fragments to an immunogenic carrier molecule renders them more immunogenic through what is commonly known as the “carrier effect”.

Carrier molecules include, e.g., proteins and natural or synthetic polymeric compounds such as polypeptides, polysaccharides, lipopolysaccharides etc. Protein carrier molecules are especially preferred, including, but not limited to, keyhole limpet hemocyanin and mammalian serum proteins such as human or bovine gammaglobulin, human, bovine or rabbit serum albumin, or methylated or other derivatives of such proteins. Other protein carriers will be apparent to those skilled in the art. Preferably, the protein carrier will be foreign to the host animal in which antibodies against the fragments are to be elicited.

Covalent coupling to the carrier molecule can be achieved using methods well known in the art, the exact choice of which will be dictated by the nature of the carrier molecule used. When the immunogenic carrier molecule is a protein, the fragments of the invention can be coupled, e.g., using water-soluble carbodiimides such as dicyclohexylcarbodiimide or glutaraldehyde.

Coupling agents, such as these, can also be used to cross-link the fragments to themselves without the use of a separate carrier molecule. Such cross-linking into aggregates can also increase immunogenicity. Immunogenicity can also be increased by the use of known adjuvants, alone or in combination with coupling or aggregation.

Adjuvants for the vaccination of animals include, but are not limited to, Adjuvant 65 (containing peanut oil, mannide monooleate and aluminum monostearate); Freund's complete or incomplete adjuvant; mineral gels such as aluminum hydroxide, aluminum phosphate and alum; surfactants such as hexadecylamine, octadecylamine, lysolecithin, dimethyldioctadecylammonium bromide, N,N-dioctadecyl-N′,N′-bis(2-hydroxymethyl) propanediamine, methoxyhexadecylglycerol and pluronic polyols; polyanions such as pyran, dextran sulfate, poly IC, polyacrylic acid and carbopol; peptides such as muramyl dipeptide, dimethylglycine and tuftsin; and oil emulsions. The polypeptides could also be administered following incorporation into liposomes or other microcarriers.

Information concerning adjuvants and various aspects of immunoassays are disclosed, e.g., in the series by P. Tijssen, Practice and Theory of Enzyme Immunoassays, 3rd Edition, 1987, Elsevier, New York. Other useful references covering methods for preparing polyclonal antisera include Microbiology, 1969, Hoeber Medical Division, Harper and Row; Landsteiner, Specificity of Serological Reactions, 1962, Dover Publications, New York, and Williams, et al., Methods in Immunology and Immunochemistry, Vol. 1, 1967, Academic Press, New York.

The anti-NPC1L1 antibody molecules of the invention preferably recognize human, mouse or rat NPC1L1; however, the present invention includes antibody molecules which recognize NPC1L1 from any species, preferably mammals (e.g., cat, sheep or horse). The present invention also includes complexes comprising an NPC1L1 polypeptide of the invention and an anti-NPC1L1 antibody molecule. Such complexes can be made by simply contacting the antibody molecule with its cognate polypeptide.

Various methods may be used to make the antibody molecules of the invention. Human antibodies can be made, for example, by methods which are similar to those disclosed in U.S. Pat. Nos. 5,625,126; 5,877,397; 6,255,458; 6,023,010 and 5,874,299.

Hybridoma cells which produce the monoclonal anti-NPC1L1 antibodies may be produced by methods which are commonly known in the art. These methods include, but are not limited to, the hybridoma technique originally developed by Kohler, et al., (1975) (Nature 256:495-497), as well as the trioma technique (Hering, et al., (1988) Biomed. Biochim. Acta. 47:211-216 and Hagiwara, et al., (1993) Hum. Antibod. Hybridomas 4:15), the human B-cell hybridoma technique (Kozbor, et al., (1983) Immunology Today 4:72 and Cote, et al., (1983) Proc. Natl. Acad. Sci. U.S.A 80:2026-2030), and the EBV-hybridoma technique (Cole, et al., in Monoclonal Antibodies and Cancer Therapy, Alan R. Liss, Inc., pp. 77-96, 1985). ELISA may be used to determine if hybridoma cells are expressing anti-NPC1L1 antibodies.

The anti-NPC1L1 antibody molecules of the present invention may also be produced recombinantly (e.g., in an E.coli/T7 expression system as discussed above). In this embodiment, nucleic acids encoding the antibody molecules of the invention (e.g., V_{H} or V_{L}) may be inserted into a pet-based plasmid and expressed in the E. coli/T7 system. There are several methods by which to produce recombinant antibodies which are known in the art. An example of a method for recombinant production of antibodies is disclosed in U.S. Pat. No. 4,816,567. See also Skerra, A., et al., (1988) Science 240:1038-1041; Better, M., et al., (1988) Science 240:1041-1043 and Bird, R. E., et al., (1988) Science 242:423-426.

The term “monoclonal antibody,” includes an antibody obtained from a population of substantially homogeneous antibodies, i.e., the individual antibodies comprising the population are identical except for possible, naturally occurring mutations that may be present in minor amounts. Monoclonal antibodies are highly specific, being directed against a single antigenic site. Monoclonal antibodies are advantageous in that they may be synthesized by a hybridoma culture, essentially uncontaminated by other immunoglobulins. The modifier “monoclonal” indicates the character of the antibody as being obtained from a substantially homogeneous population of antibodies, and is not to be construed as requiring production of the antibody by any particular method. The monoclonal antibodies to be used in accordance with the present invention may be made by the hybridoma method as described by Kohler, et al., (1975) Nature 256:495.

The term “polyclonal antibody” includes an antibody which was produced among or in the presence of one or more other, non-identical antibodies. In general, polyclonal antibodies are produced from a B-lymphocyte in the presence of several other B-lymphocytes which produced non-identical antibodies. Typically, polyclonal antibodies are obtained directly from an immunized animal (e.g., a rabbit).

A “bispecific antibody” comprises two different antigen binding regions which bind to distinct antigens. Bispecific antibodies, as well as methods of making and using the antibodies, are conventional and very well known in the art.

Anti-idiotypic antibodies or anti-idiotypes are antibodies directed against the antigen-combining region or variable region (called the idiotype) of another antibody molecule. As disclosed by Jerne (Jerne, N. K., (1974) Ann. Immunol. (Paris) 125c:373 and Jerne, N. K., et al., (1982) EMBO 1:234), immunization with an antibody molecule expressing a paratope (antigen-combining site) for a given antigen (e.g., NPC1L1) will produce a group of anti-antibodies, some of which share, with the antigen, a complementary structure to the paratope. Immunization with a subpopulation of the anti-idiotypic antibodies will, in turn, produce a subpopulation of antibodies or immune cell subsets that are reactive to the initial antigen.

The term “fully human antibody” refers to an antibody which comprises human immunoglobulin sequences only. Similarly, “mouse antibody” refers to an antibody which comprises mouse immunoglobulin sequences only.

“Human/mouse chimeric antibody” refers to an antibody which comprises a mouse variable region (V_{H} and V_{L}) fused to a human constant region.

“Humanized” anti-NPC1L1 antibodies are also within the scope of the present invention. Humanized forms of non-human (e.g., murine) antibodies are chimeric immunoglobulins, which contain minimal sequence derived from non-human immunoglobulin. For the most part, humanized antibodies are human immunoglobulins (recipient antibody) in which residues from a complementary determining region of the recipient are replaced by residues from a complementary determining region of a nonhuman species (donor antibody), such as mouse, rat or rabbit, having a desired specificity, affinity and capacity. In some instances, Fv framework residues of the human immunoglobulin are also replaced by corresponding non-human residues.

“Single-chain Fv” or “sFv” antibody fragments include the V_{H} and/or V_{L} domains of an antibody, wherein these domains are present in a single polypeptide chain. Generally, the sFv polypeptide further comprises a polypeptide linker between the V_{H} and V_{L} domains which enables the sFv to form the desired structure for antigen binding. Techniques described for the production of single chain antibodies (U.S. Pat. Nos. 5,476,786; 5,132,405 and 4,946,778) can be adapted to produce anti-NPC1L1 specific, single chain antibodies. For a review of sFv see Pluckthun in The Pharmacology of Monoclonal Antibodies, vol. 113, Rosenburg and Moore eds. Springer-Verlag, N.Y., pp. 269-315 (1994).

“Disulfide stabilized Fv fragments” and “dsFv” include molecules having a variable heavy chain (V_{H}) and/or a variable light chain (V_{L}) which are linked by a disulfide bridge.

Antibody fragments within the scope of the present invention also include F(ab)₂ fragments which may be produced by enzymatic cleavage of an IgG by, for example, pepsin. Fab fragments may be produced by, for example, reduction of F(ab)₂ with dithiothreitol or mercaptoethylamine.

An Fv fragment is a V_{L} or V_{H} region.

Depending on the amino acid sequences of the constant domain of their heavy chains, immunoglobulins can be assigned to different classes. There are at least five major classes of immunoglobulins: IgA, IgD, IgE, IgG and IgM, and several of these may be further divided into subclasses (isotypes), e.g., IgG-b 1, IgG-2, IgG-3 and IgG-4; IgA-1 and IgA-2.

The anti-NPC1L1 antibody molecules of the invention may also be conjugated to a chemical moiety. The chemical moiety may be, inter alia, a polymer, a radionuclide or a cytotoxic factor. Preferably, the chemical moiety is a polymer which increases the half-life of the antibody molecule in the body of a subject. Suitable polymers include, but are by no means limited to, polyethylene glycol (PEG) (e.g., PEG with a molecular weight of 2kDa, 5 kDa, 10 kDa, 12 kDa, 20 kDa, 30 kDa or 40 kDa), dextran and monomethoxypolyethylene glycol (mPEG). Methods for producing PEGylated anti-IL8 antibodies which are described in U.S. Pat. No. 6,133,426 can be applied to the production of PEGylated anti-NPC1L1 antibodies of the invention. Lee, et al., (1999) (Bioconj. Chem. 10:973-981) discloses PEG conjugated single-chain antibodies. Wen, et al., (2001) (Bioconj. Chem. 12:545-553) discloses conjugating antibodies with PEG which is attached to a radiometal chelator (diethylenetriaminpentaacetic acid (DTPA)).

The antibody molecules of the invention may also be conjugated with labels such as ⁹⁹Tc, ⁹⁰Y, ¹¹¹In, ³²P, ¹⁴C, ¹²⁵I, ³H, ¹³¹I, ¹¹C, ¹⁵O, ¹³N, ¹⁸F, ³⁵S, ⁵¹Cr, ⁵⁷To, ²²⁶Ra, ⁶⁰Co, ⁵⁹ Fe, ⁵⁷Se, ¹⁵²Eu, ⁶⁷CU, ²¹⁷Ci, ²¹¹At, ²¹²Pb, ⁴⁷Sc, ¹⁰⁹Pd, ²³⁴Th, ⁴⁰K, ¹⁵⁷Gd, ⁵⁵Mn, ⁵²Tr, or ⁵⁶Fe.

The antibody molecules of the invention may also be conjugated with fluorescent or chemilluminescent labels, including fluorophores such as rare earth chelates, fluorescein and its derivatives, rhodamine and its derivatives, isothiocyanate, phycoerythrin, phycocyanin, allophycocyanin, o-phthaladehyde, fluorescamine, ¹⁵²Eu, dansyl, umbelliferone, luciferin, luminal label, isoluminal label, an aromatic acridinium ester label, an imidazole label, an acridimium salt label, an oxalate ester label, an aequorin label, 2,3-dihydrophthalazinediones, biotin/avidin, spin labels and stable free radicals.

The antibody molecules may also be conjugated to a cytotoxic factor such as diptheria toxin, Pseudomonas aeruginosa exotoxin A chain, ricin A chain, abrin A chain, modeccin A chain, alpha-sarcin, Aleurites fordii proteins and compounds (e.g., fatty acids), dianthin proteins, Phytoiacca americana proteins PAPI, PAPII, and PAP-S, momordica charantia inhibitor, curcin, crotin, saponaria officinalis inhibitor, mitogellin, restrictocin, phenomycin, and enomycin.

Any method known in the art for conjugating the antibody molecules of the invention to the various moieties may be employed, including those methods described by Hunter, et al., (1962) Nature 144:945; David, et al., (1974) Biochemistry 13:1014; Pain, et al., (1981) J. Immunol. Meth. 40:219; and Nygren, J., (1982) Histochem. and Cytochem. 30:407.

Methods for conjugating antibodies are conventional and very well known in the art.

### Screening Assays

The invention allows the discovery of selective agonists and antagonists of NPC1L1 (e.g., SEQ ID NO: 2, 4 or 12) that may be useful in treatment and management of a variety of medical conditions including elevated serum sterol (e.g., cholesterol) or 5α-stanol. Thus, NPC1L1 of this invention can be employed in screening systems to identify agonists or antagonists. Essentially, these systems provide methods for bringing together NPC1L1, an appropriate, known ligand or agonist or antagonist, including a sterol (e.g., cholesterol, phytosterols (including, but not limited to, sitosterol, campesterol, stigmasterol and avenosterol)), a 5α-stanol (including but not limited to cholestanol, 5α-campesterol and 5α-sitostanol), a 5α-azetidinone (e.g., ezetimibe), BODIPY-ezetimibe (Altmann, et al., (2002) Biochim. Biophys. Acta 1580(1):77-93) or 4″, 6″-bis[(2-fluorophenyl)carbamoyl]-beta-D-cellobiosyl derivative of 11-ketotigogenin as described in DeNinno, et al., (1997) (J. Med. Chem. 40(16):2547-54) (Merck; L-166,143) or any substituted azetidinone, and a sample to be tested for the presence of an NPC1L1 agonist or antagonist.

Non-limiting examples of suitable azetidinones include those disclosed in U.S. Pat. Nos. Re. 37,721; 5,631,365; 5,767,115; 5,846,966; 5,688,990; 5,656,624; 5,624,920; 5,698,548 and 5,756,470 and U.S. patent application Publication No. 2003/0105028—each of which is herein incorporated by reference in its entirety.

A convenient method by which to evaluate whether a sample contains an NPC1L1 agonist or antagonist is to determine whether the sample contains a substance which competes for binding between the known agonist or antagonist (e.g., ezetimibe) and NPC1L1.

Ezetimibe can be prepared by a variety of methods well know to those skilled in the art, for example such as are disclosed in U.S. Pat. Nos. 5,631,365, 5,767,115, 5,846,966, 6,207,822, U.S. patent appplication Publication No. 2002/0193607 and PCT Patent Application WO 93/02048, each of which is incorporated herein by reference in its entirety.

“Sample”, “candidate compound” or “candidate substance” refers to a composition which is evaluated in a test or assay, for example, for the ability to agonize or antagonize NPC1L1 (e.g., SEQ ID NO: 2, 4 or 12) or a functional fragment thereof. The composition may small molecules, peptides, nucleotides, polynucleotides, subatomic particles (e.g., a particles, β particles) or antibodies.

Two basic types of screening systems can be used, a labeled-ligand binding assay (e.g., direct binding assay or scintillation proximity assay (SPA)) and a “sterol (e.g., cholesterol) or 5α-stanol uptake” assay. A labeled ligand for use in the binding assay can be obtained by labeling a sterol (e.g., cholesterol) or a 5α-stanol or a known NPC1L1 agonist or antagonist with a measurable group (e.g., ¹²⁵I or ³H). Various labeled forms of sterols (e.g., cholesterol) or 5α-stanols are available commercially or can be generated using standard techniques (e.g., Cholesterol- [1,2-³H(N)], Cholesterol-[1,2,6,7-³H(N)] or Cholesterol-[7-³H(N)]; American Radiolabeled Chemicals, Inc; St. Louis, Mo.). In a preferred embodiment, ezetimibe is fluorescently labeled with a BODIPY group (Altmann, et al., (2002) Biochim. Biophys. Acta 1580(1):77-93) or labeled with a detectable group such as ¹²⁵I or ³H.

Direct Binding Assay. Typically, a given amount of NPC1L1 of the invention (e.g., SEQ ID NO: 2, 4 or 12) is contacted with increasing amounts of labeled ligand or known antagonist or agonist (discussed above) and the amount of the bound, labeled ligand or known antagonist or agonist is measured after removing unbound, labeled ligand or known antagonist or agonist by washing. As the amount of the labeled ligand or known agonist or antagonist is increased, a point is eventually reached at which all receptor binding sites are occupied or saturated. Specific receptor binding of the labeled ligand or known agonist or antagonist is abolished by a large excess of unlabeled ligand or known agonist or antagonist.

Preferably, an assay system is used in which non-specific binding of the labeled ligand or known antagonist or agonist to the receptor is minimal. Non-specific binding is typically less than 50%, preferably less than 15%, and more preferably less than 10% of the total binding of the labeled ligand or known antagonist or agonist.

A nucleic acid encoding an NPC1L1 polypeptide of the invention (e.g., SEQ ID NO: 2, 4 or 12) can be transfected into an appropriate host cell, whereby the receptor will become incorporated into the membrane of the cell. A membrane fraction can then be isolated from the cell and used as a source of the receptor for assay. Alternatively, the whole cell expressing the receptor in the cell surface can be used in an assay. Preferably, specific binding of the labeled ligand or known antagonist or agonist to an untransfected/untransformed host cell or to a membrane fraction from an untransfected/untransformed host cell will be negligible.

In principle, a binding assay of the invention could be carried out using a soluble NPC1L1 polypeptide of the invention, e.g., following production and refolding by standard methods from an E. coli expression system, and the resulting receptor-labeled ligand complex could be precipitated, e.g., using an antibody against the receptor. The precipitate could then be washed and the amount of the bound, labeled ligand or antagonist or agonist could be measured.

In the basic binding assay, the method for identifying an NPC1L1 agonist or antagonist includes:

(a) contacting NPC1L1 (e.g., SEQ ID NO: 2 or 4 or 12) or a subsequence thereof, in the presence of a known amount of labeled sterol (e.g., cholesterol) or 5α-stanol or known antagonist or agonist (e.g., labeled ezetimibe or labeled L-166,143) with a sample to be tested for the presence of an NPC1L1 agonist or antagonist; and

(b) measuring the amount of labeled sterol (e.g., cholesterol) or 5α-stanol or known antagonist or agonist bound to the receptor.

An NPC1L1 antagonist or agonist in the sample is identified by measuring substantially reduced binding of the labeled sterol (e.g., cholesterol) or 5α-stanol or known antagonist or agonist to NPC1L1, compared to what would be measured in the absence of such an antagonist or agonist. For example, reduced binding between [³H]-cholesterol and NPC1L1 in the presence of a sample might suggest that the sample contains a substance which is competing against [³H]-cholesterol for NPC1L1 binding.

Alternatively, a sample can be tested directly for binding to NPC1L1 (e.g., SEQ ID NO: 2, 4 or 12). A basic assay of this type may include the following steps:

(a) contacting NPC1L1 (e.g., SEQ ID NO: 2 or 4 or 12) or a subsequence thereof with a labeled candidate compound (e.g., [³H]-ezetimibe); and

(b) detecting binding between the labeled candidate compound and NPC1L1.

A candidate compound which is found to bind to NPC1L1 may function as an agonist or antagonist of NPC1L1 (e.g., by inhibition of sterol (e.g., cholesterol) or 5α-stanol uptake).

SPA Assay. NPC1L1 antagonists or agonists may also be measured using scintillation proximity assays (SPA). SPA assays are conventional and very well known in the art; see, for example, U.S. Pat. No. 4,568,649. In SPA, the target of interest is immobilised to a small microsphere approximately 5 microns in diameter. The microsphere, typically, includes a solid scintillant core which has been coated with a polyhydroxy film, which in turn contains coupling molecules, which allow generic links for assay design. When a radioisotopically labeled molecule binds to the microsphere, the radioisotope is brought into close proximity to the scintillant and effective energy transfer from electrons emitted by the isotope will take place resulting in the emission of light. While the radioisotope remains in free solution, it is too distant from the scintillant and the electron will dissipate the energy into the aqueous medium and therefore remain undetected. Scintillation may be detected with a scintillation counter. In general, ³H and ¹²⁵I labels are well suited to SPA.

For the assay of receptor-mediated binding events, the lectin wheat germ agglutinin (WGA) may be used as the SPA bead coupling molecule (Amersham Biosciences; Piscataway, N.J.). The WGA coupled bead captures glycosylated, cellular membranes and glycoproteins and has been used for a wide variety of receptor sources and cultured cell membranes. The receptor is immobilized onto the WGA-SPA bead and a signal is generated on binding of an isotopically labeled ligand. Other coupling molecules which may be useful for receptor binding SPA assays include poly-L-lysine and WGA/polyethyleneimine (Amersham Biosciences; Piscataway, N.J.). See, for example, Berry, J. A., et al., (1991) Cardiovascular Pharmacol. 17 (Suppl.7): S 143-S145; Hoffman, R., et al., (1992) Anal. Biochem. 203: 70-75; Kienhus, et al., (1992) J. Receptor Research 12: 389-399; Jing, S., et al., (1992) Neuron 9: 1067-1079.

The scintillant contained in SPA beads may include, for example, yttrium silicate (YSi), yttrium oxide (YOx), diphenyloxazole or polyvinyltoluene (PVT) which acts as a solid solvent for diphenylanthracine (DPA).

SPA assays may be used to analyze whether a sample is an NPC1L1 antagonist or agonist. In these assays, a host cell which expresses NPC1L1 (e.g., SEQ ID NO: 2 or 4 or 12) on the cell surface or a membrane fraction thereof is incubated with SPA beads (e.g., WGA coated YOx beads or WGA coated YSi beads) and labeled, known ligand or agonist or antagonist (e.g., ³H-cholesterol, ³H-ezetimibe or ¹²⁵I-ezetimibe). The assay mixture further includes either the sample to be tested or a blank (e.g. water). After an optional incubation, scintillation is measured using a scintillation counter. An NPC1L1 agonist or antagonist may be identified in the sample by measuring substantially reduced fluorescence, compared to what would be measured in the absence of such agonist or antagonist (blank). Measuring substantially reduced fluorescence may suggest that the sample contains a substance which competes for NPC1L1 binding with the known ligand, agonist or antagonist.

Alternatively, a sample may be identified as an antagonist or agonist of NPC1L1 by directly detecting binding in a SPA assay. In this assay, a labeled version of a candidate compound to be tested may be put in contact with the host cell expressing NPC1L1 or a membrane fraction thereof which is bound to the SPA bead. Fluorescence may then be assayed to detect the presence of a complex between the labeled candidate compound and the host cell or membrane fraction expressing NPC1L1. A candidate compound which binds to NPC1L1 may possess NPC1L1 agonistic or antagonistic activity.

Host cells expressing NPC1L1 may be prepared by transforming or transfecting a nucleic acid encoding an NPC1L1 of the invention into an appropriate host cell, whereby the receptor becomes incorporated into the membrane of the cell. A membrane fraction can then be isolated from the cell and used as a source of the receptor for assay. Alternatively, the whole cell expressing the receptor on the cell surface can be used in an assay. Preferably, specific binding of the labeled ligand or known antagonist or agonist to an untransfected/untransformed host cell or membrane fraction from an untransfected/untransformed host cell will be negligible. Preferred host cells include Chinese Hamster Ovary (CHO) cells, murine macrophage J774 cells or any other macrophage cell line and human intestinal epithelial Caco2 cells.

Sterol/5α-stanol Uptake Assay. Assays may also be performed to determine if a sample can agonize or antagonize NPC1L1 mediated sterol (e.g., cholesterol) or 5α-stanol uptake. In these assays, a host cell expressing NPC1L1 (e.g., SEQ ID NO: 2 or 4 or 12) on the cell surface (discussed above) can be contacted with detectably labeled sterol (e.g., ³H-cholesterol or ¹²⁵I-cholesterol)) or 5α-stanol along with either a sample or a blank. After an optional incubation, the cells can be washed to remove unabsorbed sterol or 5α-stanol. Sterol or 5α-stanol uptake can be determined by detecting the presence of labeled sterol or 5α-stanol in the host cells. For example, assayed cells or lysates or fractions thereof (e.g., fractions resolved by thin-layer chromatography) can be contacted with a liquid scintillant and scintillation can be measured using a scintillation counter.

In these assays, an NPC1L1 antagonist in the sample may be identified by measuring substantially reduced uptake of labeled sterol (e.g., ³H-cholesterol) or 5α-stanol, compared to what would be measured in the absence of such an antagonist and an agonist may be identified by measuring substantially increased uptake of labeled sterol (e.g., ³H-cholesterol) or 5α-stanol, compared to what would be measured in the absence of such an agonist.

Mouse Assay. The present invention comprises a mutant mouse which lacks any functional NPC1L1. This mouse may serve as a convenient control experiment in screening assays for identifying inhibitors of intestinal sterol (e.g., cholesterol) or 5α-stanol absorption, preferably inhibitors of NPC1L1. Preferably, a mouse assay of the present invention would comprise the following steps:

(a) feeding a sterol (e.g., cholesterol) or 5α-stanol-containing substance (e.g., comprising radiolabeled cholesterol, such as ¹⁴C-cholesterol or ³H-cholesterol) to a first and second mouse comprising a functional NPC1L1 gene and to a third, mutant mouse lacking a functional NPC1L1;

The sterol (e.g., cholesterol) or 5α-stanol containing substance preferably contains labeled cholesterol, such as a radiolabeled cholesterol, for example, ³H or ¹⁴C labeled cholesterol. The sterol (e.g., cholesterol) or 5α-stanol containing substance may also include cold, unlabeled sterol (e.g., cholesterol) or 5α-stanol such as in corn oil.

In these assays, the third npc1l1 mutant mouse serves as a (+)-control experiment which exhibits low levels of intestinal sterol (e.g., cholesterol) or 5α-stanol absorption and the second mouse serves as a (−)-control experiment which exhibits normal, uninhibited levels of intestinal sterol (e.g., cholesterol) or 5α-stanol absorption. The second mouse is not administered the sample to be tested for an NPC1L1 antagonist. The first mouse is the experiment.

(b) administering the sample to the first mouse comprising a functional NPC1L1 but not to the second mouse;

(c) measuring the amount of sterol (e.g., cholesterol) or 5α-stanol absorption in the intestine of said first, second and third mouse;

Intestinal sterol (e.g., cholesterol) or 5α-stanol absorption may be measured by any method known in the art. For example, the level intestinal absorption can be assayed by measuring the level of serum sterol (e.g., cholesterol) or 5α-stanol .

(d) comparing the levels of intestinal sterol (e.g., cholesterol) or 5α-stanol absorption in each mouse;

wherein the sample is determined to contain the intestinal sterol (e.g., cholesterol) or 5α-stanol absorption antagonist when the level of intestinal sterol (e.g., cholesterol) or 5α-stanol absorption in the first mouse is less than the amount of intestinal sterol (e.g., cholesterol) or 5α-stanol absorption in the second mouse.

Preferably, if the sample contains an intestinal sterol (e.g., cholesterol) or 5α-stanol absorption inhibitor (e.g., an NPC1L1 inhibitor), the level of sterol (e.g., cholesterol) or 5α-stanol absorption in the first mouse will be similar to that of the third, npc1l1 mutant mouse.

An alternative, (+)-control experiment which may be used in these screening assays is a mouse comprising functional NPC1L1 which is administered a known antagonist of NPC1L1, such as ezetimibe.

### Pharmaceutical Compositions

NPC1L1 agonists and antagonists discovered, for example, by the screening methods described above may be used therapeutically (e.g., in a pharmaceutical composition) to stimulate or block the activity of NPC1L1 and, thereby, to treat any medical condition caused or mediated by NPC1L1. In addition, the antibody molecules of the invention may also be used therapeutically (e.g., in a pharmaceutical composition) to bind NPC1L1 and, thereby, block the ability of NPC1L1 to bind a sterol (e.g., cholesterol) or 5α-stanol. Blocking the binding of a sterol (e.g., cholesterol) or 5α-stanol would prevent absorption of the molecule (e.g., by intestinal cells such as enterocytes). Blocking absorption of sterol (e.g., cholesterol) or 5α-stanol would be a useful way to lower serum sterol (e.g., cholesterol) or 5α-stanol levels in a subject and, thereby, reduce the incidence of, for example, hyperlipidemia, atherosclerosis, coronary heart disease, stroke or arteriosclerosis.

The term “subject” or “patient” includes any organism, preferably animals, more preferably mammals (e.g., mice, rats, rabbits, dogs, horses, primates, cats) and most preferably humans.

The term “pharmaceutical composition” refers to a composition including an active ingredient and a pharmaceutically acceptable carrier and/or adjuvant.

Although the compositions of this invention could be administered in simple solution, they are more typically used in combination with other materials such as carriers, preferably pharmaceutically acceptable carriers. Useful, pharmaceutically acceptable carriers can be any compatible, non-toxic substances suitable for delivering the compositions of the invention to a subject. Sterile water, alcohol, fats, waxes, and inert solids may be included in a pharmaceutically acceptable carrier. Pharmaceutically acceptable adjuvants (buffering agents, dispersing agents) may also be incorporated into the pharmaceutical composition.

Preferably, the pharmaceutical compositions of the invention are in the form of a pill or capsule. Methods for formulating pills and capsules are very well known in the art. For example, for oral administration in the form of tablets or capsules, the active drug component may be combined with any oral, non-toxic pharmaceutically acceptable inert carrier, such as lactose, starch, sucrose, cellulose, magnesium stearate, dicalcium phosphate, calcium sulfate, talc, mannitol, ethyl alcohol (liquid forms) and the like. Moreover, when desired or needed, suitable binders, lubricants, disintegrating agents and coloring agents may also be incorporated in the mixture. Suitable binders include starch, gelatin, natural sugars, corn sweeteners, natural and synthetic gums such as acacia, sodium alginate, carboxymethylcellulose, polyethylene glycol and waxes. Among the lubricants there may be mentioned for use in these dosage forms, boric acid, sodium benzoate, sodium acetate, sodium chloride, and the like. Disintegrants include starch, methylcellulose, guar gum and the like. Sweetening and flavoring agents and preservatives may also be included where appropriate.

The pharmaceutical compositions of the invention may be administered in conjunction with a second pharmaceutical composition or substance. In preferred embodiments, the second composition includes a cholesterol-lowering drug. When a combination therapy is used, both compositions may be formulated into a single composition for simultaneous delivery or formulated separately into two or more compositions (e.g., a kit).

The formulations may conveniently be presented in unit dosage form and may be prepared by any methods well known in the art of pharmacy. See, e.g., Gilman et al. (eds.) (1990), The Pharmacological Bases of Therapeutics, 8th Ed., Pergamon Press; and Remington's Pharmaceutical Sciences, supra, Easton, Pa.; Avis et al. (eds.) (1993) Pharmaceutical Dosage Forms: Parenteral Medications Dekker, New York; Lieberman et al. (eds.) (1990) Pharmaceutical Dosage Forms: Tablets Dekker, New York; and Lieberman et al. (eds.) (1990), Pharmaceutical Dosage Forms: Disperse Systems Dekker, New York.

The dosage regimen involved in a therapeutic application may be determined by a physician, considering various factors which may modify the action of the therapeutic substance, e.g., the condition, body weight, sex and diet of the patient, the severity of any infection, time of administration, and other clinical factors. Often, treatment dosages are titrated upward from a low level to optimize safety and efficacy. Dosages may be adjusted to account for the smaller molecular sizes and possibly decreased half-lives (clearance times) following administration.

An “effective amount” of an antagonist of the invention may be an amount that will detectably reduce the level of intestinal sterol (e.g., cholesterol) or 5α-stanol absorption or detectably reduce the level of serum sterol (e.g., cholesterol) or 5α-stanol in a subject administered the composition.

Typical protocols for the therapeutic administration of such substances are well known in the art. Pharmaceutical composition of the invention may be administered, for example, by any parenteral or non-parenteral route.

Pills and capsules of the invention can be administered orally. Injectable compositions can be administered with medical devices known in the art; for example, by injection with a hypodermic needle.

Injectable pharmaceutical compositions of the invention may also be administered with a needleless hypodermic injection device; such as the devices disclosed in U.S. Pat. Nos. 5,399,163; 5,383,851; 5,312,335; 5,064,413; 4,941,880; 4,790,824 or 4,596,556.

### Anti-Sense

The present invention also encompasses anti-sense oligonucleotides capable of specifically hybridizing to mRNA encoding NPC1L1 (e.g., any of SEQ ID NOs: 1, 3, 5-11 or 13) having an amino acid sequence defined by, for example, SEQ ID NO: 2 or 4 or 12 or a subsequence thereof so as to prevent translation of the mRNA. Additionally, this invention contemplates anti-sense oligonucleotides capable of specifically hybridizing to the genomic DNA molecule encoding NPC1L1, for example, having an amino acid sequence defined by SEQ ID NO: 2 or 4 or 12 or a subsequence thereof.

This invention further provides pharmaceutical compositions comprising (a) an amount of an oligonucleotide effective to reduce NPC1L1-mediated sterol (e.g., cholesterol) or 5α-stanol absorption by passing through a cell membrane and binding specifically with mRNA encoding NPC1L1 in the cell so as to prevent its translation and (b) a pharmaceutically acceptable carrier capable of passing through a cell membrane. In an embodiment, the oligonucleotide is coupled to a substance that inactivates mRNA. In another embodiment, the substance that inactivates mRNA is a ribozyme.

Reducing the level of NPC1L1 expression by introducing anti-sense NPC1L1 RNA into the cells of a patient is a useful method reducing intestinal sterol (e.g., cholesterol) or 5α-stanol absorption and serum cholesterol in the patient.

### Kits

Kits of the present invention include ezetimibe, preferably combined with a pharmaceutically acceptable carrier, in a pharmaceutical formulation, more preferably in a pharmaceutical dosage form such as a pill, a powder, an injectable liquid, a tablet, dispersible granules, a capsule, a cachet or a suppository. See for example, Gilman et al. (eds.) (1990), The Pharmacological Bases of Therapeutics, 8th Ed., Pergamon Press; and Remington's Pharmaceutical Sciences, supra, Easton, Pa.; Avis et al. (eds.) (1993) Pharmaceutical Dosage Forms: Parenteral Medications Dekker, New York; Lieberman et al. (eds.) (1990) Pharmaceutical Dosage Forms: Tablets Dekker, New York; and Lieberman et al. (eds.) (1990), Pharmaceutical Dosage Forms: Disperse Systems Dekker, New York. Preferably, the dosage form is a Zetia® tablet (Merck/Schering-Plough Corp.). Ezetimibe may be supplied in any convenient form. For example, tablets including ezetimibe may be supplied in bottles of 30, 90 or 500.

The kits of the present invention also include information, for example in the form of a package insert, indicating that the target of ezetimibe is NPC1L1 (NPC3). The term “target of ezetimibe” indicates that ezetimibe reduces intestinal sterol (e.g., cholesterol) or 5α-stanol absorption, either directly or indirectly, by antagonizing NPC1L1. The form of the insert may take any form, such as paper or on electronic media such as a magnetically recorded medium (e.g., floppy disk) or a CD-ROM.

The package insert may also include other information concerning the pharmaceutical compositions and dosage forms in the kit. Generally, such information aids patients and physicians in using the enclosed pharmaceutical compositions and dosage forms effectively and safely. For example, the following information regarding ezetimibe (e.g., Zetia®) and/or simvastatin (e.g., Zocor®) may be supplied in the insert: pharmacokinetics, pharmacodynamics, clinical studies, efficacy parameters, indications and usage, contraindications, warnings, precautions, adverse reactions, overdosage, proper dosage and administration, how supplied, proper storage conditions, references and patent information.

The kits of the invention may also include simvastatin (

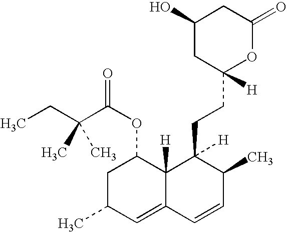

) preferably combined with a pharmaceutically acceptable carrier, in a pharmaceutical formulation, more preferably in a pharmaceutical dosage form such as a pill, a powder, an injectable liquid, a tablet, dispersible granules, a capsule, a cachet or a suppository. Preferably, the dosage form of simvastatin is a Zocor® tablet (Merck & Co.; Whitehouse Station, N.J.).

Tablets or pills comprising simvastatin may be supplied in any convenient form. For example, pills or tablets comprising 5 mg simvastatin can be supplied as follows: bottles of 30, 60, 90, 100 or 1000. Pills or tablets comprising 10 mg simvastatin may be supplied as follows: bottles of 30, 60, 90, 100, 1000 or 10,000. Pills or tablets comprising 20 mg simvastatin may be supplied as follows: bottles of 30, 60, 90, 100, 1000 or 10,000. Pills or tablets comprising 40 mg simvastatin may be supplied as follows: bottles of 30, 60, 90, 100 or 1000. Pills or tablets comprising 80 mg simvastatin may be supplied as follows: bottles of 30, 60, 90, 100, 1000 or 10,000.

Ezetimibe and simvastatin may be supplied, in the kit, as separate compositions or combined into a single composition. For example, ezetimibe and simvastatin may be supplied within a single, common pharmaceutical dosage form (e.g., pill or tablet) as in separate pharmaceutical dosage forms (e.g., two separate pills or tablets).

### EXAMPLES

The following examples are provided to more clearly describe the present invention and should not be construed to limit the scope of the invention in any way.

### Example 1

### Cloning and Expression of Rat, Mouse and Human NPC1L1

Rat NPC, mouse NPC1L1 or human NPC1L1 can all conveniently be amplified using polymerase chain reaction (PCR). In this approach, DNA from a rat, mouse or human cDNA library can be amplified using appropriate primers and standard PCR conditions. Design of primers and optimal amplification conditions constitute standard techniques which are commonly known in the art.

An amplified NPC1L1 gene may conveniently be expressed, again, using methods which are commonly known in the art. For example, NPC1L1 may be inserted into a pET-based plasmid vector (Stratagene; La Joola, Calif.), downstream of the T7 RNA polymerase promoter. The plasmid may then be transformed into a T7 expression system (e.g., BL21DE3 E.coli cells), grown in a liquid culture and induced (e.g., by adding IPTG to the bacterial culture).

### Example 2

### Direct Binding Assay

Membrane preparation: Caco2 cells transfected with an expression vector containing a polynucleotide encoding NPC1L1 (e.g., SEQ ID NO: 2, 4 or 12) are harvested by incubating in 5 mM EDTA/phosphate-buffered saline followed by repeated pipeting. The cells are centrifuged 5 min at 1000×g. The EDTA/PBS is decanted and an equal volume of ice-cold 50 mM Tris-HCl, pH 7.5 is added and cells are broken up with a Polytron (PT10 tip, setting 5, 30 sec). Nuclei and unbroken cells are sedimented at 1000×g for 10 min and then the supernatant is centrifuged at 50,000×g for 10 min. The supernatant is decanted, the pellet is resuspended by Polytron, a sample is taken for protein assay (bicinchoninic acid, Pierce), and the tissue is again centrifuged at 50,000×g. Pellets are stored frozen at −20° C.

Binding assay: For saturation binding, four concentrations of [³H]-ezetimibe (15 Ci/mmol) are incubated without and with 10⁻⁵ M ezetimibe in triplicate with 50 μg of membrane protein in a total volume of 200 μl of 50 mM Tris-HCl, pH 7.5, for 30 min at 30° C. Samples are filtered on GF/B filters and washed three times with 2 ml of cold Tris buffer. Filters are dried in a microwave oven, impregnated with Meltilex wax scintillant, and counted at 45% efficiency. For competition binding assays, five concentrations of a sample are incubated in triplicate with 18 nM [³H]-ezetimibe and 70 μg of membrane protein under the conditions described above. Curves are fit to the data with Prism (GraphPad Software) nonlinear least-squares curve-fitting program and Kᵢ values are derived from IC₅₀ values according to Cheng and Prusoff (Cheng, Y. C., et al., (1973) Biochem. Pharmacol. 22:3099-3108).

### Example 3

### SPA Assay

For each well of a 96 well plate, a reaction mixture of 10 μg human, mouse or rat NPC1L1-CHO overexpressing membranes (Biosignal) and 200 μg/well YSi-WGA-SPA beads (Amersham) in 100 μl is prepared in NPC1L1 assay buffer (25 mM HEPES, pH 7.8,2 mM CaCl₂, 1 mM MgCl₂, 125 mM NaCl, 0.1% BSA). A 0.4 nM stock of ligand-[¹²⁵I]-ezetimibe- is prepared in the NPC1L1 assay buffer. The above solutions are added to a 96-well assay plate as follows: 50 μl NPC1L1 assay buffer, 100 μl of reaction mixture, 50 μl of ligand stock (final ligand concentration is 0.1 nM). The assay plates are shaken for 5 minutes on a plate shaker, then incubated for 8 hours before cpm/well are determined in Microbeta Trilux counter (PerkinElmer).

These assays will indicate that [¹²⁵I]-ezetimibe binds to the cell membranes expressing human, mouse or rat NPC1L1. Similar results will be obtained if the same experiment is performed with radiolabeled cholesterol (e.g., ¹²⁵I-cholesterol).

### Example 4

### Cholesterol Uptake Assay

CHO cells expressing either SR-B1 or three different clones of rat NPC1L1 or one clone of mouse NPC1L1 were starved overnight in cholesterol free media then dosed with [³H]-cholesterol in a mixed synthetic micelle emulsion for 4 min, 8 min, 12 min or 24 min in the absence or presence of 10 μM ezetimibe. The cells were harvested and the lipids were organically extracted. The extracted lipids were spotted on thin-layer chromatography (TLC) plates and resolved within an organic vapor phase. The free cholesterol bands for each assay were isolated and counted in a scintillation counter.

The SR-B1 expressing cells exhibited an increase in [³H]-cholesterol uptake as early as 4 min which was also inhibited by ezetimibe. The three rat clones and the one mouse clone appeared to give background levels of [³H]-cholesterol uptake which was similar to that of the untransformed CHO cell.

These experiments will yield data demonstrating that CHO cells can perform mouse, rat and human NPC1L1-dependent uptake of [³H]-cholesterol when more optimal experimental conditions are developed.

### Example 5

### Expression of Rat NPC1L1 in Wistar Rat Tissue

In these experiments, the expression of rat NPC1L1 mRNA, in several rat tissues, was evaluated. The tissues evaluated were esophagus, stomach, duodenum, jejunum, ileum, proximal colon, distal colon, liver, pancreas, heart, aorta, spleen, lung, kidney, brain, muscle, testes, ovary, uterus, adrenal gland and thyroid gland. Total RNA samples were isolated from at least 3 male and 3 female animals and pooled. The samples were then subjected to real time quantitative PCR using Taqman analysis using standard dual-labeled fluorogenic oligonucleotide probes. Typical probe design incorporated a 5′ reporter dye (e.g., 6FAM (6-carboxyfluorescein) or VIC) and a 3′ quenching dye (e.g., TAMRA (6-carboxytetramethyl-rhodamine)).

| rat NPC1L1: | | |
|---|---|---|
| Forward: | TCTTCACCCTTGCTCTTTGC | (SEQ ID NO: 14) |
| Reverse: | AATGATGGAGAGTAGGTTGAGGAT | (SEQ ID NO: 15) |
| Probe: | [6FAM]TGCCCACCTTTGTTGTCTGCTACC[TAMRA] | (SEQ ID NO: 16) |
| rat β-actin: | | |
| Forward: | ATCGCTGACAGGATGCAGAAG | (SEQ ID NO: 17) |
| Reverse: | TCAGGAGGAGCAATGATCTTGA | (SEQ ID NO: 18) |
| Probe: | [VIC]AGATTACTGCCCTGGCTCCTAGCACCAT[TAMRA] | (SEQ ID NO: 19) |

PCR reactions were run in 96-well format with 25 μl reaction mixture in each well containing: Platinum SuperMix (12.5 μl), ROX Reference Dye (0.5 ul), 50 mM magnesium chloride (2 μl), cDNA from RT reaction (0.2 μl). Multiplex reactions contained gene specific primers at 200 nM each and FAM labeled probe at 100 nM and gene specific primers at 100 nM each and VIC labeled probe at 50 nM. Reactions were run with a standard 2-step cycling program, 95° C. for 15 sec and 60° C. for 1 min, for 40 cycles.

The highest levels of expression were observed in the duodenum, jejunum and ileum tissue. These data indicate that NPC1L1 plays a role in cholesterol absorption in the intestine.

### Example 6

### Expression of Mouse NPC1L1 in Mouse Tissue

In these experiments, the expression of mouse NPC1L1 mRNA, in several tissues, was evaluated. The tissues evaluated were adrenal gland, BM, brain, heart, islets of langerhans, LI, small intestine, kidney, liver, lung, MLN, PLN, muscle, ovary, pituitary gland, placenta, Peyers Patch, skin, spleen, stomach, testes, thymus, thyroid gland, uterus and trachea. Total RNA samples were isolate from at least 3 male and 3 female animals and pooled. The samples were then subjected to real time quantitative PCR using Taqman analysis using the following primers and probes:

mouse NPC1L1:

| | | |
|---|---|---|
| Forward: | ATCCTCATCCTGGGCTTTGC | (SEQ ID NO: 20) |
| Reverse: | GCAAGGTGATCAGGAGGTTGA | (SEQ ID NO: 21) |
| Probe: | [6FAM]CCCAGCTTATCCAGATTTTCTTCTTCCGC[TAMRA] | (SEQ ID NO: 22) |

The highest levels of expression were observed in the Peyer's Patch, small intestine, gall bladder and stomach tissue. These data are consistent with a cholesterol absorption role for NPC1L1 which takes place in the digestive system.

### Example 7

### Expression of Human NPC1L1 in Human Tissue

In these experiments, the expression level of human NPC1L1 mRNA was evaluated in 2045 samples representing 46 normal tissues. Microarray-based gene expression analysis was performed on the Affryetrix HG-U95 GeneChip using a cRNA probe corresponding to base pairs 4192-5117 (SEQ ID NO: 43) in strict accordance to Affymetrix's established protocols. Gene Chips were scanned under low photo multiplier tube (PMT), and data were normalized using either Affymetrix MAS 4.0 or MAS 5.0 algorithms. In addition “spike ins” for most samples were used to construct a standard curve and obtain RNA concentration values according Gene Logic algorithms and procedures. A summary of these results are indicated, below, in Table 2.

TABLE 2
Expression level of NPC1L1 mRNA in various human tissues.

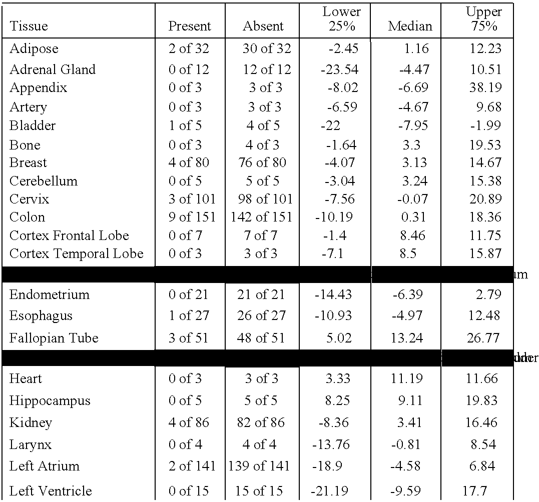

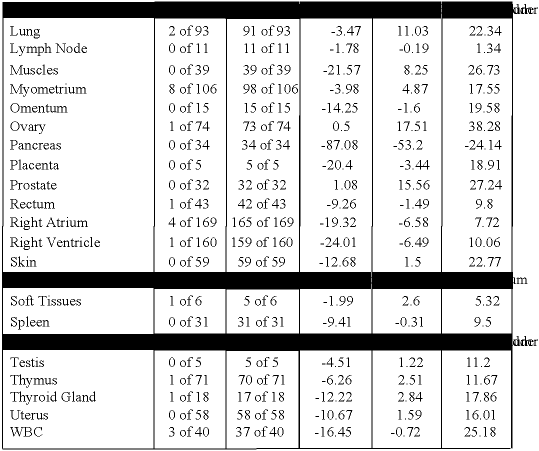

Shaded data corresponds to tissues wherein the highest levels of NPC1L1 mRNA was detected. The “Present” column indicates the proportion of specified tissue samples evaluated wherein NPC1L1 mRNA was detected. The “Absent” column indicates the proportion of specified tissue samples evaluated wherein NPC1L1 RNA was not detected. The “lower 25%”, “median” and “upper 75%” columns indicate statistical distribution of the relative NPC1L1 signal intensities observed for each set of tissue evaluated.

### Example 8

### Distribution of Rat NPC1L1, Rat IBAT or Rat SR-B1 mRNA in Rat Small Intestine

In these experiments, the distribution of rat NPC1L1 mRNA along the proximal-distal axis of rat small intestines was evaluated. Intestines were isolated from five independent animals and divided into 10 sections of approximately equal length. Total RNA was isolated and analyzed, by real time quantitative PCR using Taqman analysis, for localized expression levels of rat NPC1L1, rat IBAT (ileal bile acid transporter) or rat SR-B1 mRNA. The primers and probes used in the analysis were:

| rat NPC1L1: | | |
|---|---|---|
| Forward: | TCTTCACCCTTGCTCTTTGC | (SEQ ID NO: 23) |
| Reverse: | AATGATGGAGAGTAGGTTGAGGAT | (SEQ ID NO: 24) |
| Probe: | [6FAM]TGCCCACCTTTGTTGTCTGCTACC[TAMRA] | (SEQ ID NO: 25) |
| rat Villin: | | |
| Forward: | AGCACCTGTCCACTGAAGATTTC | (SEQ ID NO: 26) |
| Reverse: | TGGACGCTGAGCTTCAGTTCT | (SEQ ID NO: 27) |
| Probe: | [VIC]CTTCTCTGCGCTGCCTCGATGGAA[TAMRA] | (SEQ ID NO: 28) |
| rat SR-B1: | | |
| Forward: | AGTAAAAAGGGCTCGCAGGAT | (SEQ ID NO: 29) |
| Reverse: | GGCAGCTGGTGACATCAGAGA | (SEQ ID NO: 30) |
| Probe: | [6FAM]AGGAGGCCATGCAGGCCTACTCTGA[TAMRA] | (SEQ ID NO: 31) |
| rat IBAT: | | |
| Forward: | GAGTCCACGGTCAGTCCATGT | (SEQ ID NO: 32) |
| Reverse: | TTATGAACAACAATGCCAAGCAA | (SEQ ID NO: 33) |
| Probe: | [6FAM]AGTCCTTAGGTAGTGGCTTAGTCCCTGGAAGCTC[TAMRA] | (SEQ ID NO: 34) |

The mRNA expression levels of each animal intestinal section were analyzed separately, then the observed expression level was normalized to the observed level of villin mRNA in that intestinal section. The observed, normnalized mRNA expression levels for each section where then averaged.

The expression level of NPC1L1 and SR-B1 were highest in the jejunum (sections 2-5) as compared to that of the more distal ileum sections. Since the jejunum is believed to be the site of cholesterol absorption, these data suggest such a role for rat NPC1L1. IBAT distribution favoring the ileum is well document and served as a control for the experiment.

### Example 9

### In situ Analysis of Rat NPC1L1 mRNA in Rat Jejunum Tissue

The localization of rat NPC1L1 mRNA was characterized by in situ hybridization analysis of rat jejunum serial sections. The probes used in this analysis were:

| | | |
|---|---|---|
| T7-sense probe: | GTAATACGACTCACTATAGGGCCCTGACGGTCCTTCCTGA | (SEQ ID NO: 35) |
| GGGAATCTTCAC | | |
| T7-antisense probe: | GTAATACGACTCACTATAGGGCCTGGGAAGTTGGTCAT | (SEQ ID NO: 36) |
| GGCCACTCCAGC | | |

The RNA probes were synthesized using T7 RNA polymerase amplification of a PCR amplified DNA fragment corresponding rat NPC1L1 nucleotides 3318 to 3672 (SEQ ID NO 1). Sense and anti-sense digoxigenin-UTP labeled cRNA probes were generated from the T7 promoter using the DIG RNA Labeling Kit following the manufacturer's instructions. Serial cryosections rat jejunum were hybridized with the sense and antiisense probes. Digoxigenin labeling was detected with the DIG Nucleic Acid Detection Kit based on previous methods. A positive signal is characterized by the deposition of a red reaction product at the site of hybridization.

The anti-sense probe showed strong staining of epithelium along the crypt-villus axis under low magnification (40×). The observed rat NPC1L1 mRNA expression levels may have been somewhat greater in the crypts than in the villus tips. Under high magnification (200×), staining was observed in the enterocytes but not in the goblet cells. A lack of staining observed with the sense probe (control) confirmed the high specificity of the NPC1L1 anti-sense signal. These data provided further evidence of the role of rat NPC1L1 in intestinal cholesterol absorption.

### Example 10

### FACS Analysis of Fluorescently Labeled Ezetimibe Binding to Transiently Transfected CHO Cells

In these experiments, the ability of BODIPY-labeled ezetimibe (Altmann, et al., (2002) Biochim. Biophys. Acta 1580(1):77-93) to bind to NPC1L1 and SR-B1 was evaluated. “BODIPY” is a fluorescent group which was used to detect the BODIPY-ezetimibe. Chinese hamster ovary (CHO) cells were transiently transfected with rat NPC1L1 DNA (rNPC1L1/CHO), mouse NPC1L1 DNA (mNPC1L1/CHO), mouse SR-B1 DNA (mSRBI/CHO) or EGFP DNA (EGFP/CHO). EGFP is enhanced green fluorescent protein which was used as a positive control. The transfected CHO cells or untransfected CHO cells were then stained with 100 nM BODIPY-labeled ezetimibe and analyzed by FACS. Control experiments were also performed wherein the cells were not labeled with the BODIPY-ezetimibe and wherein untransfected CHO cells were labeled with the BODIPY-ezetimibe.

No staining was observed in the untransfected CHO, rNPC1L1/CHO or mNPC1L1/CHO cells. Fluorescence was detected in the positive-control EGFP/CHO cells. Staining was also detected in the mouse SR-B1/CHO cells. These data show that, under the conditions tested, BODIPY-ezetimibe is capable of binding to SR-B1 and that such binding is not ablated by the presence of the fluorescent BODIPY group. When more optimal conditions are determined, BODIPY-ezetimibe will be shown to label the rNPC1L1/CHO and mNPC1L1/CHO cells.

### Example 11

### FACS Analysis of Transiently Transfected CHO Cells Labeled with Anti-FLAG Antibody M2

In these experiments, the expression of FLAG-tagged NPC1L1 on CHO cells was evaluated. CHO cells were transiently transfected with mouse NPC1L1 DNA, rat NPC1L1 DNA, FLAG-rat NPC1L1 DNA or FLAG-mouse NPC1L1 DNA. The 8 amino acid FLAG tag used was DYKDDDDK (SEQ ID NO: 37) which was inserted on the amino-terminal extracellular loop just past the secretion signal sequence. The cells were incubated with commercially available anti-FLAG monoclonal mouse antibody M2 followed by a BODIPY-tagged anti-mouse secondary antibody. The treated cells were then analyzed by FACS.

The M2 antibody stained the CHO cells transfected with FLAG-rat NPC1L1 DNA and with FLAG-mouse NPC1L1. No staining was observed in the CHO cells transfected with mouse NPC1L1 DNA and with rat NPC1L1 DNA. These data showed that rat NPC1L1 and mouse NPC1L1 possess no significant, inherent fluorescence and are not bound by the anti-FLAG antibody. The observed, FLAG-dependent labeling of the cells indicated that the FLAG-mouse NPC1L1 and FLAG-rat NPC1L1 proteins are localized at the cell membrane of the CHO cells.

### Example 12

### FACS Analysis of FLAG-rat NPC1L1-EGFP Chimera in Transiently Transfected CHO Cell

In these experiments, the surface and cytoplasmic localization of rat NPC1L1 in CHO cells was evaluated. CHO cells were transiently transfected with FLAG-rat NPC1L1 DNA or with FLAG-rat NPC1L1-EGFP DNA. In these fusions, the FLAG tag is at amino-terminus of rat NPC1L1 and EGFP fusion is at the carboxy-terminus of rat NPC1L1. The cells were then stained with the M2 anti-FLAG mouse (primary) antibody followed by secondary staining with a BODIPY-labeled anti-mouse antibody. In control experiments, cells were stained with only the secondary antibody and not with the primary antibody (M2). The stained cells were then analyzed by FACS.

In a control experiment, FLAG-rat NPC1L1 transfected cells were stained with BODIPY anti-mouse secondary antibody but not with the primary antibody. The data demonstrated that the secondary, anti-mouse antibody possessed no significant specificity for FLAG-rat NPC1L1 and that the FLAG-rat NPC1L1, itself, possesses no significant fluorescence.

In another control experiment, unlabeled FLAG-rat NPC1L1-EGFP cells were FACS analyzed. In these experiments, autofluorescence of the enhanced green fluorescent protein (EGFP) was detected.

FLAG-rat NPC1L1 cells were stained with anti-FLAG mouse antibody M2 and with the BODIPY-labeled anti-mouse secondary antibody and FACS analyzed. The data from this analysis showed that the cells were labeled with the secondary, BODIPY-labeled antibody which indicated expression of the FLAG-rat NPC1L1 protein on the surface of the CHO cells.

FLAG-rat NP1L1-EGFP cells were stained with anti-FLAG mouse antibody M2 and with the BODIPY-labeled anti-mouse secondary antibody and FACS analyzed. The data from this analysis showed that both markers (BODIPY and EGFP) were present indicating surface expression of the chimeric protein. The data also indicated that a portion of the protein was located within the cells and may be associated with transport vesicles. These data supported a role for rat NPC1L1 in vesicular transport of cholesterol or protein expressed in subcellular organelles such as the rough endoplasmic reticulum.

### Example 13

### FACS Analysis and Fluorescent Microscopy of FLAG-rat NPC1L1-EGFP Chimera in a Cloned CHO Cell Line

In these experiments, the cellular localization of rat NPC1L1 was evaluated by FACS analysis and by immunohistochemistry. CHO cells were transfected with FLAG-rat NPC1L1-EGFP DNA and stained with anti-FLAG mouse antibody M2 and then with a BODIPY-labeled anti-mouse secondary antibody. In the fusion, the FLAG tag is at the amino-terminus of rat NPC1L1 and the enhanced green fluorescent protein (EGFP) tag is located at the carboxy-terminus of the rat NPC1L1. The stained cells were then analyzed by FACS and by fluorescence microscopy.

Cells transfected with FLAG-rat NPC1L1-EGFP DNA were stained with the anti-FLAG mouse antibody M2 and then with the BODIPY-labeled anti-mouse secondary antibody. FACS analysis of the cells detected both markers indicating surface expression of the chimeric protein.

FLAG-rat NPC1L1-EGFP transfected cells were analyzed by fluorescent microscopy at 63× magnification. Fluorescent microscopic analysis of the cells indicated non-nuclear staining with significant perinuclear organelle staining. Resolution of the image could not confirm the presence of vesicular associated protein. These data indicated that the fusion protein was expressed on the cell membrane of CHO cells.

### Example 14

### Generation of Polyclonal Anti-rat NPC1L1 Rabbit Antibodies

Synthetic peptides (SEQ ID NO: 39-42) containing an amino- or carboxy-terminal cysteine residue were coupled to keyhole limpet hemocyanin (KLH) carrier protein through a disulfide linkage and used as antigen to raise polyclonal antiserum in New Zealand white rabbits (range 3-9 months in age). The KLH-peptide was emulsified by mixing with an equal volume of Freund's Adjuvant, and injected into three subcutaneous dorsal sites. Prior to the 16 week immunization schedule a pre-immune sera sample was collected which was followed by a primary injection of 0.25 mg KLH-peptide and 3 scheduled booster injections of 0.1 mg KLH-peptide. Animals were bled from the auricular artery and the blood was allowed to clot and the serum was then collected by centrifugation

The anti-peptide antibody titer was determined with an enzyme linked immunosorbent assay (ELISA) with free peptide bound in solid phase (1 μg/well). Results are expressed as the reciprocal of the serum dilution that resulted in an OD₄₅₀ of 0.2. Detection was obtained using the biotinylated anti-rabbit IgG, horse radish peroxidase-streptavidin (HRP-SA) conjugate, and ABTS.

### Example 15

### FACS Analysis of Rat NPC1L1 Expression in CHO Cells Transiently Transfected with Rat NPC1L1 DNA Using Rabbit Anti-rat NPC1L1 Antisera

In these experiments, the expression of rat NPC1L1 on the surface of CHO cells was evaluated. CHO cells were transfected with rat NPC1L1 DNA, then incubated with either rabbit preimmune serum or with 10 week anti-rat NPC1L1 serum described, above, in Example 14 (i.e., A0715, A0716, A0867 or A0868). Cells labeled with primary antisera were then stained with a BODIPY-modified anti-rabbit secondary antibody followed by FACS analysis.

No antibody surface labeling was observed for any of the pre-immune sera samples. Specific cell surface labeling of rat NPC1L1 transfected cells was observed for both A0715 and A0868. Antisera A0716 and A0867 did not recognize rat NPC1L1 surface expression in this assay format. This indicates that the native, unfused rat NPC1L1 protein is expressed in the CHO cells and localized to the CHO cell membranes. Cell surface expression of NPC1L1 is consistent with a role in intestinal cholesterol absorption.

### Example 16

### FACS Analysis of CHO Cells Transiently Transfected with FLAG-Mouse NPC1L1 DNA or FLAG-rat NPC1L1 DNA or Untransfected CHO Cells Using Rabbit Anti-rat NPC1L1 Antisera

In these experiments, the expression of FLAG-mouse NPC1L1 and FLAG-rat NPC1L1 in CHO cells was evaluated. CHO cells were transiently transfected with FLAG-mouse NPC1L1 DNA or with FLAG-rat NPC1L1 DNA. The FLAG-mouse NPC1L1 and FLAG-rat NPC1L1 transfected cells were labeled with either A0801, A0802, A0715 or A0868 sera (see Example 14) or with anti-FLAG antibody, M2. The labeled cells were then stained with BODIPY-labeled anti-rabbit secondary antibody and FACS analyzed. The untransfected CHO cells were analyzed in the same manner as the transfected cell lines.

Positive staining of the untransfected CHO cells was not observed for any of the antisera tested. Serum A0801-dependent labeling of FLAG-rat NPC1L1 transfected cells was observed but such labeling of FLAG-mouse NPC1L1 transfected cells was not observed. Serum A0802-dependent labeling of FLAG-mouse NPC1L1 or FLAG-rat NPC1L1 transfected cells was not observed. Strong serum A0715-dependent labeling of FLAG-rat NPC1L1 transfected cells was observed and weak serum A0715-dependent labeling of FLAG-mouse NPC1L1 transfected cells was observed. Weak serum A0868-dependent labeling of rat NPC1L1 and mouse NPC1L1 transfected cells was observed. Strong Anti-FLAG M2 antibody-dependent labeling of FLAG-rat NPC1L1 and FLAG-mouse NPC1L1 transfected cells was observed. The strong M2 staining is likely to be due to the fact that M2 is an affinity-purified, monoclonal antibody of known concentration. In contrast, the respective antisera are polyclonal, unpurified and contain an uncertain concentration of anti-rat NPC1L1 antibody. These date provide further evidence that the FLAG-mouse NPC1L1 and FLAG-rat NPC1L1 proteins are expressed in CHO cells and localized to the CHO cell membranes. Cell surface expression of NPC1L1 is consistent with a role in intestinal cholesterol absorption.

### Example 17

### Immunohistochemical Analysis of Rat Jejunum Tissue with Rabbit Anti-Rat NPC1L1 Antisera A0715

In these experiments, the localization of rat NPC1L1 in rat jejunum was analyzed by immunohistochemistry. Rat jejunum was removed, immediately embedded in O.C.T. compound and frozen in liquid nitrogen. Sections (6 μm) were cut with a cryostat microtome and mounted on glass slides. Sections were air dried at room temperature and then fixed in Bouin's fixative. Streptavidin-biotin-peroxidase immunostaining was carried out using Histostain-SP kit. Endogenous tissue peroxidase activity was blocked with a 10 minute incubation in 3% H₂O₂ in methanol, and nonspecific antibody binding was minimized by a 45 minute incubation in 10% nonimmune rabbit serum. Sections were incubated with a rabbit anti-rat NPC1L1 antisera A0715 or A0868 at a 1:500 dilution at 4° C., followed by incubation with biotinylated goat anti-rabbit IgG and with streptavidin-peroxidase. Subsequently, the sections were developed in an aminoethyl carbazole (AEC)-H₂O₂ staining system and counterstained with hematoxylin and examined by microscopy. A positive reaction using this protocol is characterized by the deposition of a red reaction product at the site of the antigen-antibody reaction. Nuclei appeared blue from the hematoxylin counterstain. Controls were performed simultaneously on the neighboring sections from the same tissue block. Control procedures consisted of the following: (1) substitute the primary antibody with the pre-immune serum, (2) substitute the primary antibody with the non-immune rabbit serum, (3) substitute the primary antibody with PBS, (4) substitute the second antibody with PBS.

The example shows tissue stained with anti-rat NPC1L1 sera A07 15 or with the preimmune sera analyzed at low magnification (40×) and at high magnification (200×). The A0715-stained tissue, at low magnification, showed positive, strong staining of the villi epithelial layer (enterocytes). The A0715-stained tissue at high magnification showed positive, strong staining of the enterocyte apical membranes. No staining was observed in tissue treated only with preimmune sera. Similar results were obtained with sera A0868. These data indicate that rat NPC1L1 is expressed in rat jejunum which is consistent with a role in intestinal cholesterol absorption.

### Example 18

### Labeled Cholesterol Uptake Assay

In this example, the ability of CHO cells stably transfected with rat NPC1L1 to take up labeled cholesterol was evaluated. In these assays, cholesterol uptake, at a single concentration, was evaluated in a pulse-chase experiment. The data generated in these experiments are set forth, below, in Table 3.

Cells:

A. CHO cells stably transfected with rat NPC1L1 cDNA

B. CHO background (no transfection)

Cells were seeded at 500,000 cells/ well (mL) in 12-well plates.

Procedure:

All reagents and culture plates were maintained at 37° C. unless otherwise noted.

Starve. The maintenance media (F12 HAMS, 1% Pen/Strep, 10% FCS) was removed and the cells were rinsed with serum-free HAMS media. The serum-free media was then replaced with 1 mL “starve” media (F12 HAMS, Pen/Strep, 5% lipoprotein deficient serum (LPDS).

One plate of each cell line was starved overnight. The remaining 2 plates were designated “No Starve” (see below).

Pre-Incubation. Media was removed from all plates, rinsed with serum-free HAMS and replaced with starve media for 30 minutes.

³H-Cholesterol Pulse. The following was added directly to each well.

0.5 μCi ³H-cholesterol (˜1.1×10⁶ dpm/well) in 50 μl of a mixed bile salt micelle.

4.8 mM sodium taurocholate (2.581 mg/mL)

0.6 mM sodium oleate (0.183 mg/mL)

0.25 mM cholesterol (0.1 mg/mL)

Dispersed in “starve” media by ultrasonic vibration

Final media cholesterol concentration=5 μg/mL

Labeled cholesterol pulse time points were 0, 4, 12 and 24 minutes. Triplicate wells for each treatment were prepared.

Wash. At the designated times, media was aspirated and the cells were washed once with Hobbs Buffer A (50 mM Tris, 0.9% NaCl, 0.2% BSA, pH 7.4) and once with Hobbs Buffer B (50 mM Tris, 0.9% NaCl, pH 7.4 (no BSA)) at 37° C.

Processing/Analysis. Cells were digested overnight with 0.2N NaOH, 2 mL/well at room temperature. One 1.5 mL aliquot was removed from each well, neutralized & counted for radioactivity by scintillation counting. Two additional 50 μl aliquots from all wells are assayed for total protein by the Pierce micro BCA method. The quantity of labeled cholesterol observed in the cells was normalized by the quantity of protein in the cells.

TABLE 3
Uptake of ³H-cholesterol by CHO cells transfected
with rat NPC1L1 or mouse SR-B1 or untransfected CHO cells.

| Time, min | | | | |
|---|---|---|---|---|
| After³H- | Total Cholesterol, | | Total Cholesterol, | |
| Choles- | dpm protein ± sem | | dpm/mg protein ± sem | |
| terol | NPC1L1 | CHO | NPC1L1 | CHO |
| | No Starve | | | |
| 0 | 2067 ± 46 | 4568 ± 1937 | 10754 ± 166 | 22881 ± 9230 |
| 4 | 2619 ± 130 | 2868 ± 193 | 15366 ± 938 | 15636 ± 1471 |
| 12 | 2868 ± 193 | 4459 ± 170 | 15636 ± 1471 | 24622 ± 966 |
| 24 | 7010 ± 89 | 7204 ± 173 | 41129 ± 685 | 39361 ± 1207 |
| | Starve | | | |
| 0 | 1937 ± 273 | 2440 ± 299 | 10909 ± 1847 | 12429 ± 1673 |
| 4 | 3023 ± 308 | 2759 ± 105 | 17278 ± 1650 | 14307 ± 781 |
| 12 | 2759 ± 105 | 4857 ± 186 | 14307 ± 781 | 26270 ± 1473 |
| 24 | 6966 ± 72 | 7344 ± 65 | 39196 ± 174 | 38381 ± 161 |

dpm = disintegrations per minute
sem = standard error of the mean

### Example 19

### Effect of Ezetimibe on Cholesterol Uptake

The effect of ezetimibe on the ability of CHO cells stably transfected with mouse or rat NPC1L1 or mouse SR-B1 to take up ³H-labeled cholesterol was evaluated in pulse-chase experiments. One cDNA clone of mouse NPC1L1 (C7) and three clones of rat NPC1L1 (C7, C17 and C21) were evaluated. The ability of CHO cells stably transfected with mouse SR-B1, mouse NPC1L1 and rat NPC1L1 to take up labeled cholesterol, in the absence of ezetimibe, was also evaluated in the pulse-chase experiments. Data generated in these experiments are set forth, below, in Tables 4 and 5. Additionally, the quantity of total cholesterol taken up by transfected and untransfected CHO cells in the presence of four different unlabeled cholesterol concentrations was also evaluated. The data from these experiments is set forth, below, in Table 6.

Cells:

A. CHO cells stably transfected with rat or mouse NPC1L1 cDNA

B. CHO background (no transfection)

C. SR-B1 transfected CHO cells

Cells seeded at 500,000 cells/well (mL) in 12-well plates.

Procedure:

All reagents and culture plates were maintained at 37° C. unless otherwise noted.

Starve. The maintenance media (F12 HAMS, 1% Pen/Strep, 10% FCS) was removed and the cells were rinsed with serum-free HAMS media. The serum-free media was then replaced with 1 mL “starve” media (F12 HAMS, Pen/Strep, 5% lipoprotein deficient serum (LPDS). The cells were then starved overnight.

Pre-Incubation/pre-dose. Media was removed from all plates and replaced with fresh starve media and preincubated for 30 minutes. Half of the wells received media containing ezetimibe (stock soln in EtOH; final conc.=10 μM).

³H-Cholesterol Pulse. The following was added directly to each well:

0.5 μCi ³H-cholesterol (˜1.1×10⁶ dpm/well) in 50 μl of a mixed bile salt micelle

4.8 mM sodium taurocholate (2.581 mg/mL)

0.6 mM sodium oleate (0.183 mg/mL)

0.25 mM cholesterol (0.1 mg/mL)

Dispersed in “starve” media by ultrasonic vibration

Final media cholesterol concentration=5 μg/mL

Labeled cholesterol pulse time points were 4, 12, 24 minutes and 4 hours. Triplicate wells were prepared for each treatment.

Wash. At designated times, media was aspirated and cells were washed once with Hobbs Buffer A (50 mM Tris, 0.9% NaCl, 0.2% bovine serum albumin (BSA), pH 7.4) and once with Hobbs Buffer B (50 mM Tris, 0.9% NaCl, pH 7.4 (no BSA)) at 37° C.

Processing/Analysis.

A. 4, 12, 24 minute time points: Cells were digested overnight with 0.2N NaOH, 2 mL/well, room temperature. One 1.5 mL aliquot was removed from each well, neutralized & counted for radioactivity by scintillation counting.

B. 4 hour time point: The digested cells were analyzed by thin-layer chromatography to determine the content of cholesterol ester in the cells.

Extracts were spotted onto TLC plates and run for 30 minutes in 2 ml hexane:isopropanol (3:2) mobile phase for 30 minutes, followed by a second run in 1 ml hexane:isopropanol (3:2) mobile phase for 15 minutes.

C. Protein determination of cell extracts. Plates containing a sample of the cell extracts were placed on orbital shaker at 120 rpm for indicated times and then extracts are pooled into 12×75 tubes. Plates were dried and NaOH (2 ml/well) added. The protein content of the samples were then determined. Two additional 50 μl aliquots from all wells were assayed for total protein by the Pierce micro BCA method. The quantity of labeled cholesterol observed in the cells was normalized to the quantity of protein in the cells.

TABLE 4
Total Cholesterol in Transfected CHO Cells in the Presence
and Absence of Ezetimibe.

| | Total Cholesterol, | | Total Cholesterol, | |
|---|---|---|---|---|
| | dpm ± sem | | dpm/mg protein ± sem | |
| Clones: | Vehicle | EZ (10 μM) | Vehicle | EZ (10 μM) |
| | 4 Min Pulse | | | |
| CHO Control | 3413 ± 417 | 3222 ± 26 | 33443 ± 4070 | 31881 ± 483 |
| SR-B1 | 14207 ± 51 | 10968 ± 821 | 118242 ± 1261 | 92474 ± 2902 |
| mNPC1L1(C7) | 4043 ± 419 | 4569 ± 222 | 30169 ± 3242 | 30916 ± 1137 |
| rNPC1L1(C21) | 3283 ± 288 | 3769 ± 147 | 23728 ± 2111 | 27098 ± 689 |
| rNPC1L1(C17) | 3188 ± 232 | 3676 ± 134 | 24000 ± 832 | 28675 ± 527 |
| rNPC1L1(C7) | 1825 ± 806 | 3268 ± 121 | 15069 ± 6794 | 27285 ± 968 |
| | 12 Min Pulse | | | |
| CHO Control | 4710 ± 246 | 4532 ± 165 | 44208 ± 2702 | 43391 ± 1197 |
| SR-B1 | 16970 ± 763 | 12349 ± 298 | 140105 ± 6523 | 98956 ± 4447 |
| mNPC1L1(C7) | 6316 ± 85 | 6120 ± 755 | 45133 ± 342 | 41712 ± 4054 |
| rNPC1L1(C21) | 5340 ± 12 | 4703 ± 231 | 40018 ± 1181 | 33985 ± 1928 |
| rNPC1L1(C17) | 4831 ± 431 | 4579 ± 257 | 37378 ± 3461 | 34063 ± 1619 |
| rNPC1L1(C7) | 4726 ± 272 | 4664 ± 63 | 39100 ± 2350 | 38581 ± 784 |
| | 24 Min Pulse | | | |
| CHO Control | 7367 ± 232 | 6678 ± 215 | 65843 ± 1281 | 61764 ± 2131 |
| SR-B1 | 39166 ± 2152 | 23558 ± 1310 | 324126 ± 11848 | 198725 ± 11713 |
| mNPC1L1(C7) | 10616 ± 121 | 9749 ± 482 | 77222 ± 1040 | 74041 ± 3670 |
| rNPC1L1(C21) | 9940 ± 587 | 8760 ± 293 | 76356 ± 9618 | 66165 ± 2181 |
| rNPC1L1(C17) | 8728 ± 721 | 8192 ± 237 | 70509 ± 5189 | 62279 ± 4352 |
| rNPC1L1(C7) | 8537 ± 148 | 7829 ± 204 | 72134 ± 1305 | 63482 ± 368 |

EZ = ezetimibe

TABLE 5
Cholesterol Ester in CHO cells in the Presence or Absence of Ezetimibe.

| | Vehicle | EZ (10 μM) | Vehicle | EZ (10 μM) |
|---|---|---|---|---|
| Clones: | 4 Hour Pulse | | | |
| | Cholesteryl Ester, | | Cholesteryl Ester, | |
| | dpm ± sem | | dpm/mg protein ± sem | |
| CHO Control | 652 ± 13 | 208 ± 9 | 5647 ± 55 | 1902 ± 87 |
| SR-B1 | 47608 ± 1292 | 9305 ± 401 | 391067 ± 14391 | 72782 ± 3181 |
| mNPC1L1(C7) | 732 ± 127 | 453 ± 118 | 4994 ± 827 | 3057 ± 776 |
| rNPC1L1(C21) | 2667 ± 90 | 454 ± 33 | 18655 ± 1032 | 3193 ± 265 |
| rNPC1L1(C17) | 751 ± 74 | 202 ± 10 | 5379 ± 481 | 1510 ± 62 |
| rNPC1L1(C7) | 462 ± 25 | 191 ± 54 | 3597 ± 193 | 1496 ± 403 |
| | Free Cholesterol, | | Free Cholesterol, | |
| | dpm ± sem | | dpm/mg protein ± sem | |
| CHO Control | 61612 ± 1227 | 56792 ± 568 | 533876 ± 17770 | 519607 ± 16203 |
| SR-B1 | 214678 ± 4241 | 194519 ± 474 | 1762873 ± 46607 | 1521341 ± 4185 |
| mNPC1L1(C7) | 79628 ± 793 | 77516 ± 1910 | 544661 ± 1269 | 523803 ± 10386 |
| rNPC1L1(C21) | 71352 ± 1343 | 69106 ± 711 | 498016 ± 8171 | 485460 ± 4410 |
| rNPC1L1(C17) | 78956 ± 3782 | 71646 ± 446 | 566456 ± 29204 | 536651 ± 7146 |
| rNPC1L1(C7) | 75348 ± 2093 | 70628 ± 212 | 586127 ± 13932 | 556855 ± 7481 |

EZ = ezetimibe

TABLE 6
Uptake of labeled cholesterol in the presence of increasing amounts of
unlabeled cholesterol.

| | | | | | Total Cholesterol, dpm/mg protein ± sem | | | |
|---|---|---|---|---|---|---|---|---|
| Cold | Total Cholesterol, dpm ± sem | | | | | | (C7) | (C21) |
| Cholesterol | CHO Control | SR-B1 | mNPC1L1(C7) | rNPC1L1(C21) | CHO Control | SR-B1 | mNPC1L1 | rNPC1L1 |
| | 24 Min Pulse | | | | | | | |
| 3 μg/mL | 12271 ± 430 | 49603 ± 2428 | 14250 ± 1628 | 10656 ± 1233 | 108936 ± 5413 | 541562 ± | 140764 ± | 94945 ± |
| | | | | | | 13785 | 14433 | 12916 |
| 10 μg/mL | 16282 ± 2438 | 79967 ± 8151 | 25465 ± 3037 | 13225 ± 4556 | 151283 ± 23345 | 880224 ± | 250985 ± | 123433 ± |
| | | | | | | 82254 | 27481 | 34092 |
| 30 μg/mL | 14758 ± 1607 | 71925 ± 3863 | 19001 ± 1530 | 13218 ± 1149 | 135109 ± 12106 | 796236 ± | 180436 ± | 111522 ± |
| | | | | | | 18952 | 12112 | 6941 |
| 100 μg/mL | 16458 ± 1614 | 58185 ± 4548 | 15973 ± 1665 | 11560 ± 1132 | 149559 ± 17977 | 630143 ± | 147717 ± | 101328 ± |
| | | | | | | 3718 | 8261 | 7191 |
| | | | | | Cholesteryl Ester, dpm/mg protein ± sem | | | |
| | Cholesteryl Ester, dpm ± sem | | | | | | (C7) | (C21) |
| | CHO Control | SR-B1 | mNPC1L1(C7) | rNPC1L1(C21) | CHO Control | SR-B1 | mNPC1L1 | rNPC1L1 |
| | 4 Min Pulse | | | | | | | |
| 3 μg/mL | 2737 ± 114 | 39596 ± 1241 | 1561 ± 1 | 4015 ± 47 | 22050 ± 978 | 382641 ± | 13684 ± | 32020 ± |
| | | | | | | 5955 | 217 | 641 |
| 10 μg/mL | 1646 ± 76 | 17292 ± 362 | 998 ± 36 | 1866 ± 33 | 13323 ± 606 | 157914 ± | 8917 ± | 14849 ± |
| | | | | | | 3400 | 467 | 127 |
| 30 μg/mL | 970 ± 46 | 6642 ± 153 | 537 ± 82 | 970 ± 9 | 7627 ± 325 | 63547 ± | 4885 ± | 7741 ± |
| | | | | | | 1760 | 748 | 100 |
| 100 μg/mL | 895 ± 156 | 4777 ± 27 | 405 ± 7 | 777 ± 16 | 7135 ± 1230 | 45088 ± | 3663 ± | 6005 ± |
| | | | | | | 1526 | 68 | 198 |
| | | | | | Free Cholesterol, dpm/mg protein ± sem | | | |
| | Free Cholesterol, dpm ± sem | | | | | | (C7) | (C21) |
| | CHO Control | SR-B1 | mNPC1L1(C7) | rNPC1L1(C21) | CHO Control | SR-B1 | mNPC1L1 | rNPC1L1 |
| | 4 Min Pulse | | | | | | | |
| 3 μg/mL | 89013 ± 3724 | 211783 ± 3268 | 104343 ± 2112 | 92244 ± 987 | 717308 ± 34130 | 2047695 ± | 914107 ± | 735498 ± |
| | | | | | | 16213 | 5869 | 11209 |
| 10 μg/mL | 136396 ± 8566 | 278216 ± 10901 | 196173 ± 4721 | 125144 ± 877 | 1105118 ± 76074 | 2540130 ± | 1753072 ± | 996824 ± |
| | | | | | | 92471 | 86578 | 27850 |
| 30 μg/mL | 131745 ± 2922 | 224429 ± 2556 | 149172 ± 19689 | 117143 ± 4976 | 1036195 ± 21142 | 2149315 ± | 1357136 ± | 934772 ± |
| | | | | | | 78068 | 180264 | 43202 |
| 100 μg/mL | 79336 ± 4011 | 231470 ± 4221 | 114599 ± 2803 | 93538 ± 1588 | 632965 ± 29756 | 2182022 ± | 1035979 ± | 723225 ± |
| | | | | | | 36793 | 30329 | 21694 |
| | | | | | Cholesteryl Ester, dpm/mg protein ± sem | | | |
| | Cholesteryl Ester, dpm ± sem | | | | | | (C7) | (C21) |
| | CHO Control | SR-B1 | mNPC1L1(C7) | rNPC1L1(C21) | CHO Control | SR-B1 | mNPC1L1 | rNPC1L1 |
| | 24 Min Pulse | | | | | | | |
| 3 μg/mL | 57373 ± 2704 | 162296 ± 1644 | 22986 ± 940 | 59377 ± 953 | 357629 ± 14639 | 1248900 ± | 160328 ± | 401315 ± |
| | | | | | | 18565 | 6565 | 5557 |
| 10 μg/mL | 33730 ± 1296 | 112815 ± 373 | 14836 ± 552 | 31797 ± 525 | 215004 ± 5942 | 830231 ± | 98594 ± | 200451 ± |
| | | | | | | 12764 | 4205 | 5239 |
| 30 μg/mL | 19193 ± 100 | 58668 ± 1413 | 8878 ± 355 | 18963 ± 380 | 122071 ± 1271 | 446581 ± | 59091 ± | 119728 ± |
| | | | | | | 3472 | 2697 | 2131 |
| 100 μg/mL | 16761 ± 398 | 31280 ± 1270 | 8784 ± 946 | 14933 ± 311 | 103235 ± 1739 | 272796 ± | 60670 ± | 96215 ± |
| | | | | | | 13392 | 4597 | 1023 |
| | | | | | Free Cholesterol, dpm/mg protein ± sem | | | |
| | Free Cholesterol, dpm ± sem | | | | | | (C7) | (C21) |
| | CHO Control | SR-B1 | mNPC1L1(C7) | rNPC1L1(C21) | CHO Control | SR-B1 | mNPC1L1 | rNPC1L1 |
| | 24 Min Pulse | | | | | | | |
| 3 μg/mL | 248985 ± 4207 | 357819 ± 4519 | 285610 ± 5187 | 227244 ± 1016 | 1552637 ± 18954 | 2752957 ± | 1993256 ± | 1536023 ± |
| | | | | | | 24984 | 56968 | 10304 |
| 10 μg/mL | 231208 ± 8927 | 269822 ± 5872 | 311777 ± 8227 | 231666 ± 6198 | 1477414 ± 85954 | 1984473 ± | 2069980 ± | 1461157 ± |
| | | | | | | 18420 | 25517 | 58517 |
| 30 μg/mL | 203566 ± 6008 | 225273 ± 5932 | 279604 ± 6612 | 209372 ± 3386 | 1294878 ± 41819 | 1716066 ± | 1859476 ± | 1321730 ± |
| | | | | | | 52581 | 29507 | 5452 |
| 100 μg/mL | 178424 ± 2379 | 167082 ± 2211 | 229832 ± 4199 | 182678 ± 7709 | 1099648 ± 25160 | 1455799 ± | 1599244 ± | 1177546 ± |
| | | | | | | 9885 | 76938 | 51191 |

### Example 20

### Labeled Cholesterol Uptake Assay

In this example, the ability of CHO cells transiently transfected with rat NPC1L1 or mouse SR-B1 to take up labeled cholesterol was evaluated. Also evaluated was the ability of rat NPC1L1 to potentiate the ability of CHO cells transfected with mouse SR-B1 to take up labeled cholesterol. In these assays, cholesterol uptake, at a single concentration, was evaluated in pulse-chase experiments. The data generated in these experiments are set forth, below, in Table 7.

Cells:

A. CHO background cells (mock transfection).

B. CHO cells transiently transfected with mouse SR-B1.

C. CHO transiently transfected with rat NPC1L1 cDNAs (n=8 clones).

Transiently transfected cells were seeded at 300,000 cells/well (mL) in 12-well plates.

Procedure:

All reagents and culture plates were maintained at 37° C. unless otherwise noted.

Starve. The maintenance media (F12 HAMS, 1% Pen/Strep, 10% FCS) was removed from the cells and replaced with 1 mL “starve” media (F12 HAMS, Pen/Strep, 5% lipoprotein deficient serum (LPDS). Cells were starved for 1 hour.

³H-Cholesterol Pulse. The following was added directly to each well.

0.5 μCi ³H-cholesterol (˜1.1×10⁶ dpm/well) in 50 μl of a mixed bile salt micelle.

4.8 mM sodium taurocholate (2.581 mg/mL)

0.6 mM sodium oleate (0.183 mg/mL)

0.25 mM cholesterol (0.1 mg/mL)

Dispersed in “starve” media by ultrasonic vibration

Final media cholesterol concentration=5 μg/mL

Labeled cholesterol pulse time points were 24 Min and 4 hours. Triplicate wells for each treatment.

Wash. At the designated times, media was aspirated and cells were washed once with Hobbs Buffer A (50 mM Tris, 0.9% NaCl, 0.2% BSA, pH 7.4) and once with Hobbs Buffer B (50 mM Tris, 0.9% NaCl, pH 7.4 (no BSA)) at 37° C.

Proeessing/Analysis.

A. 24 minute time point: Cells were digested overnight with 0.2N NaOH, 2 mL/well at room temp. One, 1.5 mL aliquot was removed from each well, neutralized & counted for radioactivity by scintillation counting.

B. 4 hour time point: The digested cells were analyzed by thin-layer chromatography to determine the content of cholesterol ester in the cells.

The extracts were spotted onto thin layer chromatography plates and run in 2 ml hexane:isopropanol (3:2) containing mobile phase for 30 minutes, followed by a second run in 1 ml hexane:isopropanol (3:2) containing mobile phase for 15 min.

C. Protein determination of cell extracts: Plates containing a sample of the cell extracts were placed on orbital shaker at 120 rpm for indicated times and then extracts are pooled into 12×75 tubes. Plates were dried and NaOH (2 ml/well) added. The protein content of the samples were then determined. Two additional 50 μl aliquots from all wells were assayed for total protein by the Pierce micro BCA method. The quantity of labeled cholesterol observed in the cells was normalized to the quantity of protein in the cells.

TABLE 7
Labeled cholesterol uptake in transiently transfected CHO cells.

| | |
|---|---|
| dpm | dpm/mg protein |

| | |
|---|---|
| | Total Cholesterol, ± sem |
| | 24 Min Pulse |
| Transfection | |

| | | |
|---|---|---|
| CHO Control (mock) | 4721 ± 436 | 49024 ± 4328 |
| SR-BI (Transient) | 5842 ± 82 | 59445 ± 1099 |
| NPC1L1 (Transient) | 4092 ± 377 | 47026 ± 2658 |
| SR-BI/NPC1L1 (trans) | 3833 ± 158 | 52132 ± 3071 |

Cholesteryl Ester, ± sem
4 Hour Pulse

| | | |
|---|---|---|
| CHO Control (mock) | 2132 ± 40 | 20497 ± 640 |
| SR-BI (Transient) | 5918 ± 237 | 51812 ± 1417 |
| NPC1L1 (Transient) | 1944 ± 93 | 19788 ± 642 |
| SR-BI/NPC1L1 (trans) | 4747 ± 39 | 58603 ± 1156 |

Free Cholesterol, ± sem
4 Hour Pulse

| | | |
|---|---|---|
| CHO Control (mock) | 45729 ± 328 | 439346 ± 5389 |
| SR-BI (Transient) | 50820 ± 2369 | 444551 ± 9785 |
| NPC1L1 (Transient) | 39913 ± 1211 | 406615 ± 6820 |
| SR-BI/NPC1L1 (trans) | 37269 ± 1225 | 459509 ± 6195 |

### Example 21

### Expression of Rat, Mouse and Human NPC1L1

In this example, NPC1L1 was introduced into cells and expressed. Species specific NPC1L1 expression constructs were cloned into the plasmid pCDNA3 using clone specific PCR primers to generate the ORF flanked by appropriate restriction sites compatible with the polylinker of the vector. For all three species of NPC1L1, small intestine total tissue RNA was used as a template for reverse transcriptase-polymerase chain reaction (RT-PCR) using oligo dT as the template primer. The rat NPC1L1 was cloned as an EcoRI fragment, human NPC1L1 was cloned as a XbaI/NotI fragment and mouse NPC1L1 was cloned as an EcoRI fragment. Forward and reverse strand sequencing of each clone was performed to confirm sequence integrity. Standard transient transfection procedures were used with CHO cells. In a 6-well plate CHO cells were plated 1 day before transfection at a plating density of 2×10⁵ cells/well. The following day, cells were incubated with 2 μg plasmid DNA and 6 μL Lipofectamine for 5 hours followed a fresh media change. Forty-eight hours later, cells were analyzed for NPC1L1 expression using anti-NPC1L1 antisera by either FACS or western blot. To establish stable long term cell lines expressing NPC1L1, transfected CHO cells were selected in the presence of geneticin (G418, 0.8 mg/ml) as recommended by the manufacturer (Life Technologies). Following one month of selection in culture, the cell population was stained with anti-NPC1L1 antisera and sorted by FACS. Individual positive staining cells were cloned after isolation by limiting dilution and then maintained in selective media containing geneticin (0.5 mg/ml).

Other cell types less susceptible to transfection procedures have been generated using adenoviral vector systems. This system used to express NPC1L1 is dervied from Ad 5, a type C adenovirus. This recombinant replication-defective adenoviral vector is made defective through modifications of the E1, E2 and E4 regions. The vector also has additional modifications to the E3 region generally affecting the E3b region genes RIDa and RIDb. NPC1L1 expression was driven using the CMV promoter as an expression cassette substituted in the E3 region of the adenovirus. Rat and mouse NPC1L1 were amplified using clone specific primers flanked by restriction sites compatible with the adenovirus vector Adenovirus infective particles were produced from 293-D22 cells in titers of 5×10¹⁰ P/mL. Viral lysates were used to infect cells resistant to standard transfection methodologies. In Caco2 cells, which are highly resistant to heterologous protein expression, adenovirus mediated expression of NPC1L1 has been shown by western blot analysis to persist at least 21 days post-infection.

### Example 22

### NPC1L1 Knock-Out Transgenic Mouse

NPC1L1 knockout mice were constructed via targeted mutagenesis. This methodology utilized a targeting construct designed to delete a specific region of the mouse NPC1L1 gene. During the targeting process the E. coli lacZ reporter gene was inserted under the control of the endogenous NPC1L1 promoter. The region in NPC1L1 (SEQ ID NO: 45) being deleted is from nucleotide 790 to nucleotide 998. The targeting vector contains the LacZ-Neo cassette flanked by 1.9 kb 5′ arm ending with nucleotide 789 and a 3.2 kb 3′ arm starting with nucleotide 999. Genomic DNA from the recombinant embryonic stem cell line was assayed for homologous recombination using PCR. Amplified DNA fragments were visualized by agarose gel electrophoresis. The test PCRs employed a gene specific primer, which lies outside of and adjacent to the targeting vector arm, paired with one of three primers specific to the LacZ-Neo cassette sequence. For 5′ PCR reconfirmnation, the NPC1L1 specific oligonucleotide ATGTTAGGTGAGTCTGAACCTACCC (SEQ ID NO: 46) and for 3′ PCR reconfirmnation the NPC1L1 specific oligonucleotide GGATTGCATTTCCTTCAA GAAAGCC (SEQ ID NO: 47) were used. Genotyping of the F2 mice was performed by multiplex PCR using the NPC1L1 specific forward primer TATGGCTCTGCCC TCTGCAATGCTC (SEQ ID NO: 48) the LacZ-Neo cassette specific forward primer TCAGCAGCCTCTGTTCCACATACACTTC (SEQ ID NO: 49) in combination with the NPC1L1 gene specific reverse primer GTTCCACAGGGTCTGTGGTGAGTTC (SEQ ID NO: 50) allowed for determination of both the targeted and endogenous alleles. Analysis of the PCR products by agarose gel electrophoresis distinguished the wild-type, heterozygote and homozygote null mouse from each other.

### Example 23

### Acute Cholesterol Absorption in NPC1L1-Deficient Mice

To determine whether NPC1L1 plays a role in cholesterol absorption, NPC1L1 deficient mice were studied.

Mice deficient in NPC1L1 (−/−) were generated by breeding heterozygote mice (+/) to obtain wild-type (+/+) and NPC1L1 deficient mice (−/−). Non-fasted mice (6.5-9 weeks old, mixed 129 and C57BL/6 background) were weighed and grouped (n=2−/− and n=4 +/+). All animals were gavaged (Feeding needles, 24 G×1 inch, Popper and Sons, NY) with 0.1 ml corn oil (Sigma; St. Louis, Mo.) containing 1 μCi ¹⁴C-cholesterol (New England Nuclear, [⁴⁻¹⁴C] Cholesterol, NEC-0 18) and 0.1 mg carrier cholesterol mass (Sigma; St. Louis, Mo.). Two hours later, blood was collected by heart puncture. The liver was removed, weighed, and three samples were placed into 20 ml counting vials. Tissues were digested in 1 ml of 1N NaOH at 60° C. overnight. The tissue digests were acidified by addition of 250 μl of 4N HCl prior to liquid scintillation counting (LSC). Plasma was isolated by centrifugation at 10,000 rpm for 5 minutes in a microfuge and duplicate 100 μl aliquots of plasma were taken for LSC.

Cholesterol absorption, evaluated by this acute technique and expressed as the total amount of radioactive cholesterol in the plasma and liver, demonstrated that the wild type mice (+/+) absorbed an average of 11,773 dpm and NPC1L1 deficient mice absorbed 992 dpm of the ¹⁴C-cholesterol. These results indicate that the NPC1L1 deficient mice have a 92% reduction in cholesterol absorption. These data confirm the role of NPC1L1 in intestinal cholesterol absorption. Inhibition of NPC1L1-mediated cholesterol absorption, in a subject, by administering NPC1L1 antagonists, such as ezetimibe, to the subject, are a useful way to reduce serum cholesterol levels and the occurrence of atherosclerosis in the subject.

The present invention is not to be limited in scope by the specific embodiments described herein. Indeed, various modifications of the invention in addition to those described herein will become apparent to those skilled in the art from the foregoing description. Such modifications are intended to fall within the scope of the appended claims.

Patents, patent applications, publications, product descriptions, Genbank Accession Numbers and protocols are cited throughout this application, the disclosures of which are incorporated herein by reference in their entireties for all purposes.

We claim:

1. An isolated polypeptide comprising 42 or more contiguous amino acids from an amino acid sequence selected from SEQ ID NOs: 2 and 12.

2. An isolated polypeptide comprising an amino acid sequence selected from SEQ ID NOs: 2 and 12.

3. An isolated polynucleotide encoding a polypeptide of claim 1.

4. An isolated polynucleotide comprising a nucleotide sequence selected from SEQ ID NOs: 1 and 11.

5. A recombinant vector comprising the polynucleotide of claim 3.

6. A host cell comprising the vector of claim 5.

7. An antibody which specifically binds to a polypeptide of claim 1.

8. An antibody which specifically binds to a polypeptide comprising an amino acid sequence selected from SEQ ID NOs: 39-42.

9. A method for making a polypeptide comprising culturing a host cell of claim 6 under conditions in which the nucleic acid is expressed.

10. The method of claim 9 wherein the polypeptide is isolated from the culture.

11. A method for identifying an antagonist of NPC1L1 comprising:
(a) contacting a host cell expressing a polypeptide comprising an amino acid sequence selected from SEQ ID NOs: 2, 4 and 12 or a functional fragment thereof on a cell surface, in the presence of a known amount of detectably labeled ezetimibe, with a sample to be tested for the presence of the antagonist; and
(b) measuring the amount of detectably labeled ezetimibe specifically bound to the polypeptide;
wherein an NPC1L1 antagonist in the sample is identified by measuring substantially reduced binding of the detectably labeled ezetimibe to the polypeptide, compared to what would be measured in the absence of such an antagonist.

12. A method for identifying an antagonist of NPC1L1 comprising:
(a) placing, in an aqueous suspension, a plurality of support particles, impregnated with a fluorescer, to which a host cell expressing a polypeptide comprising an amino acid sequence selected from SEQ ID NOs: 2, 4 and 12 or a functional fragment thereof on a cell surface are attached;
(b) adding, to the suspension, radiolabeled ezetimibe and a sample to be tested for the presence of the antagonist, wherein the radiolabel emits radiation energy capable of activating the fluorescer upon the binding of the ezetimibe to the polypeptide to produce light energy, whereas radiolabeled ezetimibe that does not bind to the polypeptide is, generally, too far removed from the support particles to enable the radioactive energy to activate the fluorescer; and
(c) measuring the light energy emitted by the fluorescer in the suspension;
wherein an NPC1L1 antagonist in the sample is identified by measuring substantially reduced light energy emission, compared to what would be measured in the absence of such an antagonist.

13. The method of claim 12 wherein the fluorescer is selected from yttrium silicate, yttrium oxide, diphenyloxazole and polyvinyltoluene.

14. A method of claim 11 wherein the ezetimibe is labeled with a radiolabel selected from ³H and ¹²⁵I.

15. A method of claim 12 wherein the ezetimibe is labeled with a radiolabel selected from ³H and ¹²⁵I.

16. A method for identifying an antagonist of NPC1L1 comprising:
(a) contacting a host cell expressing a polypeptide comprising an amino acid sequence selected from SEQ ID NOs: 2, 4 and 12 or a functional fragment thereof on a cell surface with a detectably labeled sterol or 5α-stanol and with a sample to be tested for the presence of the antagonist; and
(b) measuring the amount of detectably labeled sterol or 5α-stanol in the cell;
wherein an NPC1L1 antagonist in the sample is identified by measuring substantially reduced detectably labeled sterol or 5α-stanol within the host cell, compared to what would be measured in the absence of such an antagonist.

17. The method of claim 16 wherein the sterol or 5α-stanol is detectably labeled with a radiolabel selected from ³H and ¹²⁵I.

18. The method of claim 16 wherein the sterol is cholesterol.

19. A method according to claim 11 wherein the host cell is selected from a chinese hamster ovary (CHO) cell, a J774 cell, a macrophage cell and a Caco2 cell.

20. A method according to claim 12 wherein the host cell is selected from a chinese hamster ovary (CHO) cell, a J774 cell, a macrophage cell and a Caco2 cell.

21. A method according to claim 16 wherein the host cell is selected from a chinese hamster ovary (CHO) cell, a J774 cell, a macrophage cell and a Caco2 cell.

22. A mutant mouse comprising a homozygous mutation of endogenous, chromosomal NPC1L1 wherein the mouse does not produce any functional NPC1L1 protein.

23. The mouse of claim 22 wherein the mouse exhibits a reduced serum sterol or 5α-stanol level.

24. The mouse of claim 22 wherein the region of endogenous, chromosomal NPC1L1 deleted corresponds to nucleotides 790-998 of the nucleotide sequence set forth in SEQ ID NO: 45.

25. An offspring or progeny of the mouse of claim 22 wherein the offspring or progeny has inherited a mutated NPC1L1 allele of said mouse.

26. A method for screening a sample for an intestinal sterol or 5α-stanol absorption antagonist comprising:
(a) feeding a sterol or 5α-stanol -containing substance to a first and second mouse comprising a functional NPC1L1 gene and to a third, mutant mouse of claim 21;
(b) administering the sample to the first mouse but not the second mouse;
(c) measuring the amount of sterol or 5α-stanol absorption in the intestine of said first, second and third mouse; and
(d) comparing the levels of intestinal sterol or 5α-stanol absorption in said first, second and third mouse;
wherein the sample is determined to contain the intestinal sterol or 5α-stanol absorption antagonist when the level of intestinal sterol or 5α-stanol absorption in the first mouse is less than the amount of intestinal sterol or 5α-stanol absorption in the second mouse.

27. The method of claim 26 wherein the sterol is cholesterol.

28. The method of claim 27 wherein the cholesterol is radiolabeled.

29. The method of claim 26 wherein the level of sterol or 5α-stanol cholesterol absorption is determined by measuring the level of serum sterol or 5α-stanol in the mice.

30. A method for inhibiting NPC1L1 mediated sterol or 5α-stanol uptake, in a subject, by administering, to the subject, a substance identified by the method of claim 11.

31. A method for inhibiting NPC1L1 mediated sterol or 5α-stanol uptake, in a subject, by administering, to the subject, a substance identified by the method of claim 12.

32. A method for inhibiting NPC1L1 mediated sterol or 5α-stanol uptake, in a subject, by administering, to the subject, a substance identified by the method of claim 16.

33. A method for inhibiting NPC1L1 mediated sterol or 5α-stanol uptake, in a subject, by administering, to the subject, a substance identified by the method of claim 26.

34. A kit comprising:
(a) ezetimibe in a pharmaceutical dosage form; and
(b) information indicating that NPC1L1 is a target of ezetimibe.

35. The kit of claim 34 wherein the dosage form is a tablet comprising 10 mg ezetimibe.

36. The kit of claim 34 further comprising simvastatin in a pharmaceutical dosage form.

37. The kit of claim 36 wherein the simvastatin in pharmaceutical dosage form comprises 5 mg, 10 mg, 20 mg, 40 mg or 80 mg simvastatin.

38. The kit of claim 36 wherein the simvastatin in pharmaceutical dosage form and the ezetimibe in pharmaceutical dosage form are associated in a single pill or tablet.

39. A method for decreasing the level of intestinal sterol or 5α-stanol absorption in a subject comprising reducing the level of expression of NPC1L1 in the subject.

40. The method of claim 39 wherein the subject is a mouse, rat or human.

41. The method of claim 39 wherein the level of expression of NPC1L1 in the subject is reduced by mutating NPC1L1 in the subject.

42. The method of claim 39 wherein the sterol is cholesterol.

43. A method for identifying an antagonist of NPC1L1 comprising:
(a) contacting a host cell expressing a polypeptide comprising an amino acid sequence selected from SEQ ID NOs: 2, 4 and 12 or a functional fragment thereof on a cell surface, in the presence of a known amount of a detectably labeled 2-azetidinone, with a sample to be tested for the presence of the antagonist; and
(b) measuring the amount of detectably labeled 2-azetidinone specifically bound to the polypeptide;
wherein an NPC1L1 antagonist in the sample is identified by measuring substantially reduced binding of the detectably labeled 2-azetidinone to the polypeptide, compared to what would be measured in the absence of such an antagonist.

44. A kit comprising:
(a) a 2-azetidinone in a pharmaceutical dosage form; and
(b) information indicating that NPC1L1 is a target of the 2-azetidinone.
